(19) United States
(12) Patent Application Publication
(10) Pub. No.: US 2007/0254906 A1
(43) Pub. Date: Nov. 1, 2007

(54) Method of Administration of Dopamine Receptor Agonists

(75) Inventors: Prabhavathi B. Fernandes, Chapel Hill, NC (US); Richard Bernard Mailman, Chapel Hill, NC (US); David Earl Nichols, West Lafayette, IN (US)

Correspondence Address: BARNES & THORNBURG LLP, 11 SOUTH MERIDIAN, INDIANAPOLIS, IN, 46204, US

(73) Assignee: DarPharma, Inc., Chapel Hill, NC (US)

(21) Appl. No.: 11/632,874

(22) PCT Filed: Jul. 21, 2005
(86) PCT No.: PCT/US05/26899

§ 371 Date: Jan. 19, 2007

Related U.S. Application Data
(60) Provisional application No. 60/589,764, filed on Jul. 21, 2004.

(51) Int. Cl. A61K 31/473; A61K 31/4741

(52) U.S. Cl. 514/288; 514/284

(57) ABSTRACT

Methods for treating a patient having pulmonary edema are described. The methods include administering to the lung endobronchial space of the airways of the patient an effective amount of a dopamine D₁ receptor agonist. Dopamine D₁ receptor agonists, including hexahydrobenzophenanthridine, hexahydrothienophenanthridine, phenyltetrahydrobenzazepine, chromenoisoquinoline, naphthoisoquinoline dopamine receptor agonists, and their pharmaceutically acceptable salts, formulated as aerosols and dry powders are also described.

### CROSS-REFERENCE TO RELATED APPLICATION

This application claims the priority benefit under 35 U.S.C. § 119(e) of U.S. provisional patent application Ser. No. 60/589,764, filed Jul. 21, 2004, the disclosure of which is incorporated herein in its entirety by reference.

### TECHNICAL FIELD

This invention pertains to methods for treating respiratory disorders characterized by pulmonary edema. In particular, this invention pertains to methods for treatment that include pulmonary administration.

### BACKGROUND

Pulmonary edema is a serious clinical complication that may result from a variety of infectious and non-infectious causes, including but not limited to severe acute respiratory syndrome (SARS), adult respiratory distress syndrome (ARDS), and other viral pneumonias, influenza, hantavirus pulmonary syndrome (HPS), mechanical ventilation, and others. Lung injury results from the infectious or non-infectious causes, and leads to changes in the alveolar-capillary barrier in the lungs. These changes correspond to the formation of pulmonary edema. Pulmonary edema may be characterized by the secretion of a large amount of fluid into the lung as a response to the infectious or non-infectious causes. The patient can drown in their own lung fluid before the patient has time to respond to primary treatment of the underlying disease. In patients with pulmonary edema, management of the increased fluid in the lungs is critical for patient survival. Non-treatment of pulmonary edema can lead to a significant increase in the duration, severity, and mortality rates of the underlying disease. In contrast, the clearance of edema fluid may prevent hypoxia, additional bacterial overgrowth, and/or allow penetration of effective drugs to treat the underlying disease, such as antimicrobial drugs and surfactants.

For example, SARS, caused by a newly identified coronavirus (SARS-Cov), is transmitted via secretions, allergens, or person-to person contact. While SARS originated in the Guangdong province of China, it rapidly spread worldwide due to global air travel. Symptoms are similar to flu, and include an elevated temperature, and respiratory symptoms such as cough, shortness of breath, difficulty breathing, or radiographic evidence of pneumonia. Three to 14 days after the report of the first symptoms, some patients develop a dry or non-productive cough. Current experimental treatments include the use of high doses of steroids and antiviral medications. Whereas the overall mortality rate of SARS is up to 30%, it is dramatically higher (>50%) in people >65 years of age. Management of the increased fluid in the lungs is critical for patient survival.

Further, each year influenza epidemics are responsible for a significant number of deaths. The rates of serious illness and death are highest in the >65 year of age population, and in people with increased risk for complications. In addition to the annual risk of influenza epidemics, there are infrequent yet devastating pandemics. Reported fatalities are primarily due to pulmonary edema resulting from virus-induced pulmonary capillary leak.

Another virus that results in endothelial damage and pulmonary edema is HPS. While the number of patients contracting HPS each year is small, the outbreaks caused by the disease are sporadic with high fatality. The outbreaks are disruptive and cause panic in the people in the affected area. The pulmonary capillary leak syndrome is the primary pathophysiology responsible for the cardiopulmonary and renal dysfunction experienced by patients having this disease. Current methods include only supportive treatment, such as ventilation. Approximately 33% of patients show evidence of pulmonary edema in the initial radiograph (CDC), providing an early window for treatment with a therapeutic that could clear the edema, and prevent progression to a more severe state.

Though the specific incidence of acute respiratory distress syndrome (ARDS) has been difficult to determine due to the variety of causes of this disorder, it is a common problem in intensive care units. Most often, ARDS is a progressive disorder and can be broken down into two distinct phases. Phase one, the acute phase, is manifested by rapid onset of respiratory failure in a patient with a pre-existing risk factor. In contrast, phase two is distinguished by persistent hypoxemia, increased alveolar dead space, and a further decrease in pulmonary function. The mortality rate ranges from 40-60%, depending on other contributing factors such as age, chronic liver disease, sepsis, and non-pulmonary organ dysfunction. Radiographs of phase one patients are indistinguishable from those patients suffering from pulmonary edema. Therefore, management of pulmonary edema during phase one stage may prevent the progression of ARDS to the more serious phase two.

In addition, many SARS patients progress to a stage closely resembling ARDS. For those patients, the average length of stay jumps to nearly 27 days. Even among patients who survive these severe cases, fatigue and dyspnea occur frequently, limiting their daily activities in the workplace and home. The ability to reverse ARDS-associated pulmonary edema may improve the limited success seen with conventional surfactant therapy, which is designed to improve oxygenation.

Finally, several potential bioterrorism agents are known to cause pulmonary capillary leak leading to pulmonary edema. Though the inhalation of aerosolized Staphylococcal enterotoxin B may not produce significant mortality, conventional treatment of those exposed includes only supportive care such as ventilation, or the use of vasopressors and/or diuretics. It is appreciated that an easily administered therapeutic that would lessen the duration and severity of the pulmonary edema may be most effective following the exposure of a large number of people. Similarly, chemical choking agents are also known to cause pulmonary edema. The most notorious of these is phosgene. Phosgene is a severe pulmonary irritant. Though serious pulmonary effects may be delayed up to 48 hours, phosgene poisoning may cause respiratory and cardiovascular failure, which results from an accumulation of fluid in the lungs, with secondary systemic damage as a result of anoxia.

Current management of patients in respiratory distress include mechanical ventilation, fluid management, and hemodynamic management. There is yet a need for the development of effective agents to alleviate the respiratory complications resulting from uncontrolled pulmonary edema. Studies have shown that strategies to reduce pulmonary edema lead to shorter stays in the intensive care unit and are associated with decreased mortality.

### SUMMARY OF THE INVENTION

The present invention is based on the finding that specific dopamine receptor agonists can cause clearance of pulmonary edema when administered to the lung endobronchial space of the airways of a patient. In one embodiment, the methods described herein are for treating a patient suffering from pulmonary edema caused by an infective agent such as severe acute respiratory syndrome (SARS), SARS Cov, SARS-based pneumonias, pneumonia, community acquired pneumonia, nosocomial pneumonia, other pneumonias of public health concern caused by other pathogens, or caused by toxins such as phosgene, adult respiratory distress syndrome (ARDS), ARDS of any pathology associated with pulmonary edema, influenza, hantavirus pulmonary syndrome (HPS), cystic fibrosis, primary pulmonary hypertension (PPH), secondary pulmonary hypertension (SPH), neurogenic pulmonary edema, cardiogenic pulmonary edema, toxic insults, asthma, narcotic overdose, bronchiectasis, bronchitis, in the context of Biowarfare defense, and the like, or a combination of such infective agents. In another embodiment, the methods described herein are for treating a patient suffering from pulmonary edema caused by a non-infective agent, such as ventilator induced lung injury, shocked lung, aspiration, and the like, or a combination of such non-infective agents. In another embodiment, the methods described herein are for treating a patient suffering from pulmonary edema caused by a combination of one or more infective agents and one or more non-infective agents.

The methods described herein include administering to the lung endobronchial space of the airways of the patient an effective amount of a dopamine receptor agonist. In one aspect, the dopamine receptor agonist is in the form of an aerosol or a dry powder. In another aspect, the dopamine receptor agonist is a compound selected from the group consisting of hexahydrobenzophenanthridines, hexahydrothienophenanthridines, phenylbenzodiazepines, chromenoisoquinolines, and naphthoisoquinolines, pharmaceutically acceptable salts thereof, and combinations thereof. In one embodiment, the dopamine receptor agonist is selective for dopamine receptors as compared to other monoamine receptors, including but not limited to, adrenergic receptors, serotonergic receptors, and the like. In another embodiment, the dopamine receptor agonist is selective for a D₁-like dopamine receptor subtype, such as a dopamine D₁, dopamine D_{1A}, dopamine D_{1B}, and/or a dopamine D₅ receptor, as compared to D₂-like dopamine receptor subtype, such as a dopamine D₂, a dopamine D_{2L}, a dopamine D_{2S}, a dopamine D₃, and/or a dopamine D₄ receptor. In another embodiment, the systemic absorption of the dopamine receptor agonist from the endobronchial space is insubstantial. In an alternative embodiments, varying levels, each of which is less than about 20%, of the dopamine receptor agonist administered to the patient is absorbed systemically.

In another embodiment, methods for the safe and effective removal or the enhancement of reabsorption of fluid from the lung of patients, including pneumonia, SARS, ARDS, and HPS patients is described, where such removal or enhancment provides time for the patients to respond to conventional therapies targeted at treating underlying disease states characterized by pulmonary edema. It is appreciated that the clearance of edema fluid prevents hypoxia, additional bacterial overgrowth and also allows penetration of conventional drugs, such as effective antimicrobial drugs. In another embodiment, methods for symptomatic treatment of life-threatening pulmonary edema are described.

In one illustrative embodiment, the dopamine D₁ agonist is a compound selected from the following group of compounds: 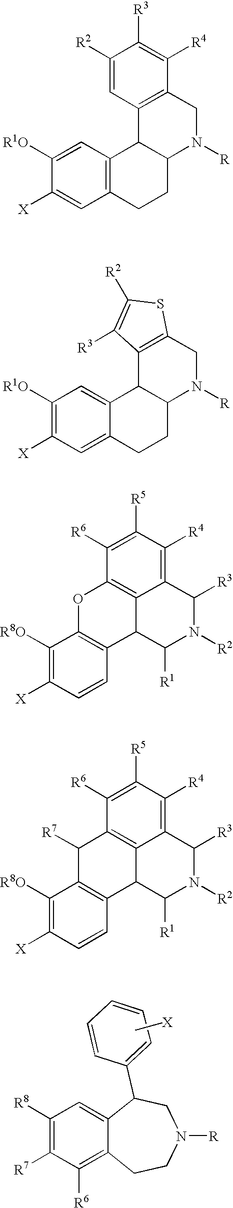
wherein, the groups R, R¹, R², R³, R⁴, R⁵, R⁶, R⁷, R⁸, and X are as defined herein.

It is appreciated that each of the foregoing compounds have one or more asymmetric carbon atoms or chiral centers, and that each may be prepared in or isolated in optically pure form, or in various mixtures of enantiomers or diastereomers. Each of the individual stereochemically pure isomers of the foregoing are contemplated herein. In addition, various mixtures of such stereochemically pure isomers are also contemplated, including but not limited to racemic mixtures that are formed from one pair of enantiomers.

In another illustrative aspect, the dopamine D₁ agonist is a compound selected from the following group of compounds: 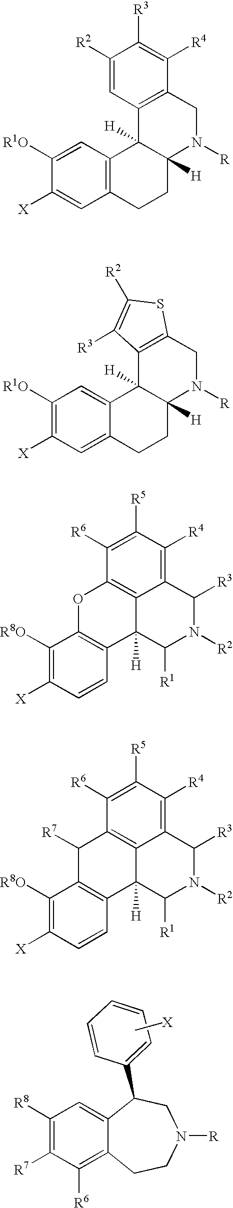
wherein, the groups R, R¹, R², R³, R⁴, R⁵, R⁶, R⁷, R⁸, and X are as defined herein, and the compounds are in optically pure form as shown, or are various mixtures of enantiomers, including racemic mixtures, of the compounds with the relative stereochemistry shown.

### BRIEF DESCRIPTION OF THE DRAWINGS

FIG. 1 illustrates the synthesis of Examples 1-4 for preparation of dihydrexidine and other hexahydrobenzo[α]phenantlridine compounds: (a) 1. Benzylamine, H₂O; 2. ArCOCl, Et₃N; (b) hv; (c) BH₃.THF; (d) H₂, 10% Pd/C; (e) 48% HBr, reflux.

FIG. 2 illustrates the synthesis of Examples 7-8 for preparation of dinoxyline and other chromeno[4,3,2-de]isoquinoline compounds: (a) 1. NaH, THF; 2. CH₃OCH₂Cl, 0° C.→r.t.; 82%; (b) 1. n-BuLi; 2. −78° C.→r.t.; 76%; (c) KNO₃, H₂SO₄; 89%; (d) Pd(Ph₃)₄, KOH, Bu₄N+Cl-, H₂0, DME, reflux; (e) TsOH.H₂O, MeOH; 98%; (f) DMF, K₂CO₃, 80° C.; 86%; (g) PtO₂, AcOH, HCl, H₂; 99%; (h) R-L, K₂CO₃, acetone; (i) BBr₃, CH₂Cl₂, −78° C.→r.t.; 72%.

FIG. 3 illustrates the synthesis of Example 9 for preparation of 2-methyl-2,3-dihydro-4(1H)-isoquinolone, an intermediate in the synthesis of dinapsoline, from ethyl 2-toluate: (a) NBS (N-bromosuccinimide, benzoylperoxide, CCl₄, reflux; (b) sarcosine ethylester HCl, K₂CO₃, acetone; (c) 1. NaOEt, EtOH, reflux, 2. HCl, reflux.

FIG. 4 illustrates the synthesis of Example 10 for preparation of dinapsoline from 2,3-dimethoxy-N,N′-diethylbenzamide: (a) 1. sec-butyllithium, TMEDA, Et₂O, −78° C., 2. Compound 20, 3. TsOH, toluene, reflux; (b) 1. 1-chloroethylchloroformate, (CH₂Cl)₂, 2. CH₃OH; (c) TsCl, Et₃N; (d) H₂, Pd/C, HOAc; (e) BH₃.THF; (f) conc. H₂SO₄, −40° C. to −5° C.; (g) Na/Hg, CH₃OH, Na₂HPO₄; (h)BBr₃, CH₂Cl₂.

FIG. 5 illustrates the asymmetric synthesis of (+)-dihydrexidine ((+)-DHX): (a) PhLi, (S,S)-1,2-dimethoxy-1,2-diphenylethane, PhCH₃, −78° C.; (b) i. NaOCH₃, PhCH₃, reflux, ii. H₂O, reflux; (c) i. HN(c-Hex)₂, EtOH, ii. recrystallization from EtOH; (d) i. ClCO₂Et, Et₃N, acetone, −5° C., ii. NaN₃, acetone, −5° C., iii. PhCH₃, reflux, iv. aq. HCl, reflux; (e) TsCl, Et₃N, CH₂Cl₂; (f) CH₂(OCH₃)₂, BF₃—OEt₂; (g) TMSOTf, CH₂Cl₂, −40 to −5° C.; (h) Na, napthalene, DME, −78° C.; (i) i. BBr₃, CH₂Cl₂, ii. HCl, EtOH.

FIG. 6 illustrates a schematic of the regulation of pulmonary fluid by the sodium potassium adenosine triphosphatase (Na/K-ATPase) pump from the apical (alveolar space) to the basolateral.

### DETAILED DESCRIPTION

Methods for treating patients in need of relief from disease states characterized by pulmonary edema are generally described herein. Without being bound by theory, it is believed that the methods provide a therapeutic benefit to the patients being treated by improving either fluid clearance, fluid resorption, or both. In particular, it is believed that methods provide a therapeutic benefit to the patients being treated by stimulating and/or increasing alveolar fluid clearance. The clearance of edema fluid may prevent hypoxia, additional bacterial overgrowth, and/or allow penetration of conventional drug for treating the underlying disease, such as antimicrobial drugs.

In one embodiment, the methods described herein are for treating a patient suffering or in need of relief from pulmonary edema caused by an infective agent such as severe acute respiratory syndrome (SARS), SARS Cov, SARS-based pneumonias, pneumonia, community acquired pneumonia, nosocomial pneumonia, other pneumonias of public health concern caused by other pathogens, or caused by toxins such as phosgene, adult respiratory distress syndrome (ARDS), ARDS of any pathology associated with pulmonary edema, influenza, hantavirus pulmonary syndrome (HPS), cystic fibrosis, primary pulmonary hypertension (PPH), secondary pulmonary hypertension (SPH), neurogenic pulmonary edema, cardiogenic pulmonary edema, toxic insults, asthma, narcotic overdose, bronchiectasis, bronchitis, in the context of Biowarfare defense, and the like, or a combination of such infective agents. In another embodiment, the methods described herein are for treating a patient suffering from pulmonary edema caused by a non-infective agent, such as ventilator induced lung injury, shocked lung, aspiration, and the like, or a combination of such non-infective agents. In another embodiment, the methods described herein are for treating a patient suffering from pulmonary edema caused by a combination of one or more infective agents and one or more non-infective agents.

In one aspect, the methods described herein are used to deliver dopamine receptor agonists in the form of an aerosol or dry powder to the lungs, including the alveoli, of a patient having pulmonary edema in amounts effective to evoke therapeutic responses in patients suffering from the disease states or disorders.

Dopamine D₁ receptors have been found to be functionally linked to physiologically and clinically relevant targets that may be used in the treatment of pulmonary edema. For example, activation of dopamine D₁ receptors mediates the clearance of pulmonary edema by upregulating the transport of subunits of sodium potassium adenosine triphosphatase (Na/K-ATPase), which in turn leads to an increase in the transport of sodium ions (Na+) and transfer of fluid from the alveoli.

Assessment of basal alveolar fluid resorption levels indicated that wild-type and transgenic mice lacking the D₁ receptor reflected similar levels of alveolar fluid resorption. Using electroporation to overexpress the D₁ receptor in the transgenic mice lacking the receptor resulted in a 50% increase in alveolar fluid resorption for these animals, compared to control animals. This increase was blocked by the D₁ antagonist SCH23390, supporting the belief that the effect was mediated through the D₁ receptor.

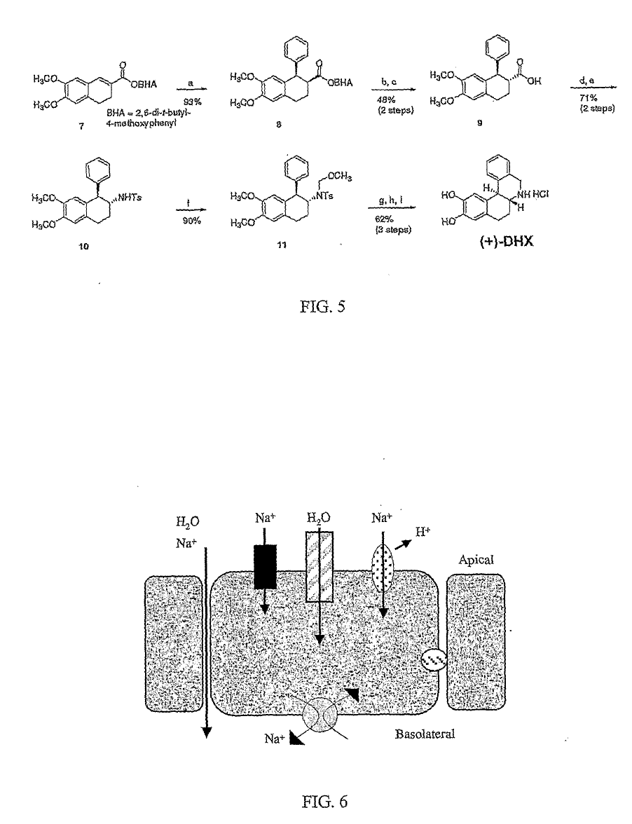

It has been shown that dopamine, when administered parenterally, causes redistribution of Na+/K+-ATPase molecules to the plasma membrane that can result in lung edema clearance. Matthay et al. in (1982) “Differential liquid and protein clearance from the alveoli of anesthetized sheep” J Appl. Physiol, 53:96-104 described that active Na+ transport from the alveolar space, across the alveolar epithelium, and into the pulmonary circulation regulated alveolar fluid resorption. The transport of Na+ ions creates an osmotic gradient, whereby water follows the Na+ ion gradient. This pathway results in clearance of fluid from the lungs. A schematic of this process is depicted in FIG. 6. Na+ ions are transported from the apical surface of the alveolar space by Na+ channels. Na+ ions are then actively transported out of the alveolar epithelium and into the pulmonary interstitium. The mechanism by which Na+ions are extruded from the basolateral surface of the lungs to the interstitium is believed to be through the Na/K-ATPase pump. See generally, Garat et al. (1997) “Alveolar epithelial fluid clearance mechanisms are intact after moderate hyperoxic lung injury in rats” Chest, 111:1381-1388; Jiang et al. (1998) “Adrenergic stimulation of Na+ transport across alveolar epithelial cells involves activation of apical Cl-channels” Am. Am. J. Physiol, 275:C1610-C1620; Lasnier et al. (1996) “Terbutaline stimulates alveolar fluid resorption in hyperoxic lung injury” J. Appl. Physiol, 81:1723-1729; Matalon et al. (1996) “Biophysical and molecular properties of amiloride-inhibitable Na+channels in alveolar epithelial cells” Am. J. Physiol, 271 :L1-22; Matthay et al. (1996) “Salt and water transport across alveolar and distal airway epithelia in the adult lung” Am. J. Physiol, 270:L487-L503; Saldias et al. (1999) “Isoproterenol improves ability of lung to clear edema in rats exposed to hyperoxia” J. Appl. Physiol, 87:30-35.

Further, dopamine and dopamine D₁ receptor agonists enhance fluid removal in animal models of both normal lung function and lung injury. See generally, Barnard et al. (1997) “Dopamine stimulates sodium transport and liquid clearance in rat lung epithelium” Am. J Respir. Crit. Care Med., 156:709-714; Barnard et al. (1999) “Stimulation of the dopamine 1 receptor increases lung edema clearance” Am. J. Respir. Crit Care Med., 160:982-986. Rats are exposed to 100% 02 for 64 hours to damage the alveolar capillary barrier and induce pulmonary edema. Following exposure, dopamine is instilled at 10-5, 10-6, 10-8. In each case, the treatments are found to increase lung liquid clearance (131%, 47%, and 27% respectively). Further, that increased clearance was blocked by SCH23390, a dopamine D₁ receptor antagonist, indicating that dopamine clears lung fluid through a dopamine D₁ receptor mediated pathway. Further, dopamine administration to isolated, perfused rat lungs increases alveolar fluid resorption by 98%, whereas the dopamine D₂ receptor agonist quinpirole does not show any effect on alveolar fluid resorption. The effect observed after dopamine administration is blocked by the dopamine D₁ selective antagonist SCH23390, but not by the dopamine D₂ antagonist sulpiride indicating that activation of dopamine D₁ receptors leads to an enhancement of lung liquid clearance.

In another embodiment, aerosol administration of the active ingredients is described. Aerosol and dry powder formulations for delivery to the lungs and devices for delivering such formulations to the endobronchial space of the airways of a patient are described in U.S. Pat. No. 6,387,886, incorporated herein by reference; and in Zeng et al., Int'l J. Pharm., vol. 191: 131-140 and Odumu et al., Pharm. Res., vol. 19: 1009-1012. It is to be understood that any other art-recognized formulations or delivery devices can be used with the compounds and methods described herein. The dopamine D₁ receptor agonist can be in the form of an aerosol or a dry powder illustratively diluted in water or saline, and the diluted solution may illustratively have a pH in the range from about 5.5 to about 7.0.

In another embodiment, a solution of the dopamine D₁ receptor agonist compounds described herein can be delivered using a nebulized aerosol formulation, nebulized by a jet, ultrasonic or electronic nebulizer, capable of producing an aerosol, illustratively with a particle size of between about 1 and about 5 microns. In another embodiment, the formulation of the dopamine D₁ receptor agonist compounds described herein can be administered in dry powder form where the active ingredient comprises part or all of the mass of the powder delivered. In one aspect, the formulation may be delivered using a dry powder or metered dose inhaler, and the like. In another aspect, the powder can have average diameters ranging from about 1 to about 5 microns formed by media milling, jet milling, spray drying, or other particle precipitation techniques.

In another embodiment, a portable therapeutic device, such as an inhaler, that used the methods described herein is described. Such a portable therapeutic may be made available to the armed services to counter a bioterrorism attack on troops or the general public, and allow on site treatment to begin before the most severe stages of the disease, with the objective of lessening the severity and mortality of the chemical or biological insult.

In one illustrative embodiment, the dopamine agonist is a compound selected from the group consisting of hexahydrobenzophenanthridines, hexahydrothienophenanthridines, phenylbenzodiazepines, chromenoisoquinolines, naphthoisoquinolines, analogs and derivatives thereof, and pharmaceutically acceptable salts thereof, including combinations of the foregoing.

In another illustrative aspect, the dopamine agonist is a compound selected from the following group of compounds: 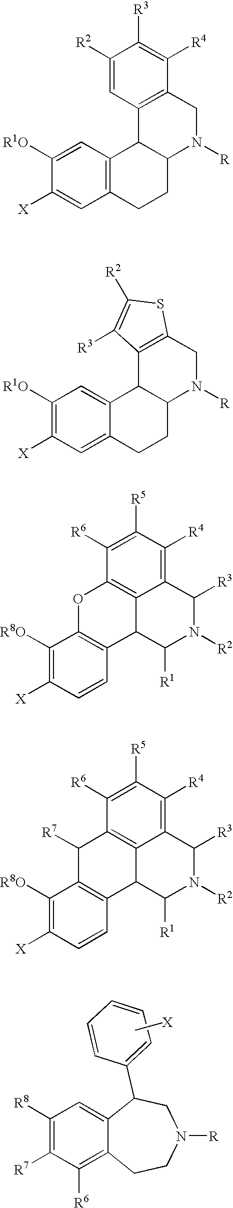
wherein, the groups R, R¹, R², R³, R⁴, R⁵, R⁶, R⁷, R⁸, and X are as defined herein.

It is appreciated that each of the foregoing compounds have one or more asymmetric carbon atoms or chiral centers, and that each may be prepared in or isolated in optically pure form, or in various mixtures of enantiomers or diastereomers. Each of the individual stereochemically pure isomers of the foregoing are contemplated herein. In addition, various mixtures of such stereochemically pure isomers are also contemplated, including but not limited to racemic mixtures that are formed from one pair of enantiomers.

In another illustrative aspect, the dopamine agonist is a compound selected from the following group of compounds: 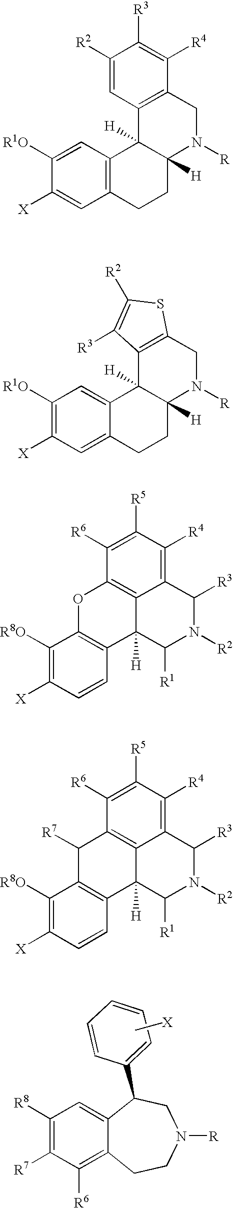
wherein, the groups R, R¹, R², R³, R⁴, R⁵, R⁶, R⁷, R⁸, and X are as defined herein, and the compounds are in optically pure form as shown, or are various mixtures of enantiomers, including racemic mixtures, of the compounds with the relative stereochemistry shown.

In another illustrative aspect, the dopamine agonist is a compound selected from the following group of compounds: 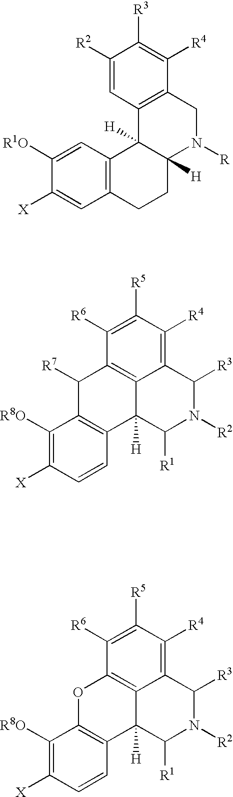
wherein, the groups R, R¹, R², R³, R⁴, R⁵, R⁶, R⁷, R⁸, and X are as defined herein, and the compounds are in optically pure form as shown, or are various mixtures of enantiomers, including racemic mixtures, of the compounds with the relative stereochemistry shown.

In one embodiment, the dopamine D₁ receptor agonist is a hexahydrobenzo[α]phenanthridine compound. Illustrative hexahydrobenzo[α]phenanthridine compounds for use in the method and composition described herein include, but are not limited to, trans-5,6,6a,7,8,12b-hexahydrobenzo[α]phenanthridine compounds of Formula I: 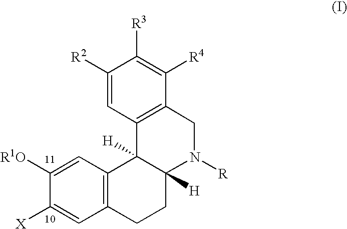
and pharmaceutically acceptable salts thereof, wherein R is hydrogen or C₁-C₄ alkyl; R¹ is hydrogen, acyl, such as C₁-C₄ alkanoyl, benzoyl, pivaloyl, and the like, an active ester group, such as a prodrug and the like, or an optionally substituted phenyl or phenoxy protecting group; X is hydrogen, fluoro, chloro, bromo, iodo or a group of the formula —OR⁵ wherein R⁵ is hydrogen, C₁-C₄ alkyl, acyl, such as C₁-C₄ alkanoyl, benzoyl, pivaloyl, and the like, an active ester group, such as a prodrug and the like, or an optionally substituted phenyl or phenoxy protecting group, provided that when X is a group of the formula —OR⁵, the groups R¹ and R⁵ can optionally be taken together to form a —CH₂— or —(CH₂)₂— group, thus representing a methylenedioxy or ethylenedioxy functional group bridging the C-10 and C-11 positions on the hexahydrobenzo[a]phenanthridine ring system; and R², R³, and R⁴ are each independently selected from hydrogen, C₁-C₄ alkyl, phenyl, fluoro, chloro, bromo, iodo, and a group —OR⁶ wherein R⁶ is hydrogen, acyl, such as C₁-C₄ alkanoyl, belizoyl, pivaloyl, and the like, an active ester group, such as a prodrug and the like, or an optionally substituted phenyl or pehnoxy protecting group; and pharmaceutically acceptable salts thereof. It is appreciated that compounds having Formula I are chiral.

As used herein, the term “acyl” refers to an optionally substituted alkyl or aryl radical connected through a carbonyl (C═O) group, such as optionally substituted alkanoyl, and optionally substituted aroyl or aryloyl. Illustrative acyl groups include, but are not limited to C₁-C₄ alkanoyl, acetyl, propionyl, butyryl, pivaloyl, valeryl, tolyl, trifluoroacetyl, anisyl, and the like.

The term “active ester group” refers to groups forming carboxylate derivatives that are hydrolyzed in vivo, under appropriately selected conditions, to the parent carboxylic acid. Such groups include prodrugs. Illustrative active ester forming groups include, but are not limited to, 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, and the like; dialkylaminoalkyl groups, such as ethoxycarbonyloxymethyl, β-ethoxycarbonyloxyethyl, and the like; dialkylaminoalkyl, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidy, dimethoxyphthalidyl, and the like.

In another embodiment, X is hydrogen, or a group of the formula —OR⁵. In another embodiment, when X in Formula I is a group of the formula —OR⁵ the groups R¹ and R⁵ can be taken together to form a —CH₂— or —(CH₂)₂— group, thus representing a methylenedioxy or ethylenedioxy functional group bridging the C-10 and C-11 positions on the hexahydrobenzo[α]phenanthridine ring system.

In another embodiment, at least one of R², R³, and R⁴ is other than hydrogen. It is appreciated that the phenoxy protecting groups used herein may diminish or block the reactivity of the nitrogen to which they are attached. In addition, the phenoxy protecting groups used herein may also serve as prodrugs, and the like. It is understood that the compounds of Formula I are chiral. It is further understood that although a single enantiomer is depicted, each enantiomer, or various mixtures of each enatiomer are contemplated as included in the methods, and compositions described herein.

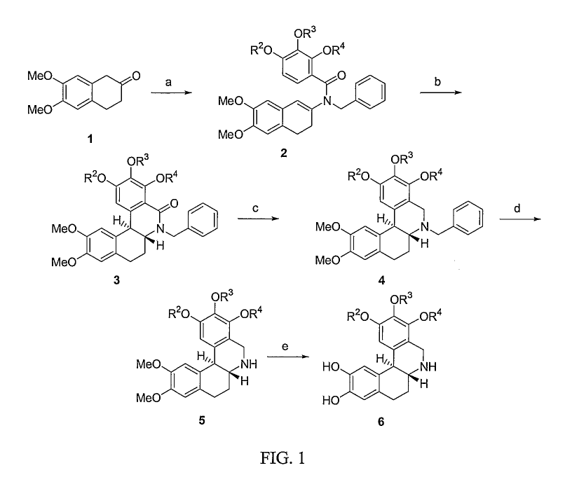

In accordance with the method and composition described herein, “C₁-C₄ alkoxy” as used herein refers to branched or straight chain alkyl groups comprising one to four carbon atoms bonded through an oxygen atom, including, but not limited to, methoxy, ethoxy, and t-butoxy. The compounds of Formula I are prepared using the same preparative chemical steps described for the preparation of the hexahydrobenzo[α]phenanthridine compounds (see FIG. 1) using the appropriately substituted benzoic acid acylating agent starting material instead of the benzoyl chloride reagent used in the initial reaction step. Thus, for example, the use of 4-methylbenzoyl chloride will yield a 2-methyl-hexahydrobenzo[α]phenanthridine compound.

In another embodiment of compounds of formula I, where X is —OR⁵, R¹ and R⁵ are different. In one aspect, one of R¹ and R⁵ is hydrogen or acetyl and the other of R¹ and R⁵ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms, and the physiologically acceptable salts thereof.

In another embodiment, the D₁ dopamine receptor agonist for use in the method and composition described herein is represented by compounds having Formula II: 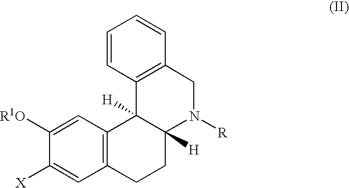
wherein R, R₁, and X are as defined in Formula I, and pharmaceutically acceptable salts thereof. It is appreciated that compounds having Formula II are chiral. It is further appreciated that although a single enantiomer is depicted, each enantiomer alone and/or various mixtures, including racemic mixtures, of each enantiomer are contemplated, and may be included in the compounds, compositions, and methods described herein.

The term “C₁-C₄ alkyl” as used herein refers to straight-chain or branched alkyl groups comprising one to four carbon atoms, such as methyl, ethyl, propyl, isopropyl, butyl, isobutyl, sec-butyl, cyclopropylmethyl, and the like. The selectivity of the compounds for the dopamine D₁ and D₂ receptors may be affected by the nature of the nitrogen substituent. Optimal dopamine D₁ agonist activity has been noted where R in formulae I-II is hydrogen or methyl. One compound of Formula II for use in the method and composition of the present invention is trans-10,11-dihydroxy-5,6,6a,7,8, 12b-hexahydrobenzo[α]phenanthridine hydrochloride, denominated hereinafter as “dihydrexidine.”

N-Alkylation may be used to prepare compounds of formula I-II wherein R is other than hydrogen, and can be effected using a variety of known synthetic methods, including, but not limited to, reductive animation of the compounds wherein R═H with an aldehyde and a reducing agent, treatment of the same with an alkyl halide, treatment with a carboxylic acid in the presence of sodium borohydride, or treatment with carboxylic acid anhydrides followed by reduction, for example with lithium aluminum hydride or with borane as the reducing agent.

All active compounds described herein bear an oxygen atom at the C-11 position as shown in formulae I-II above. The C-10 unsubstituted, C-11 hydroxy compounds possess dopamine D₁ antagonist, or weak agonist activity, depending on the alkyl group that is attached to the nitrogen atom. The more potent dopamine D₁ agonist compounds exemplified herein have a 10,11-dioxy substitution pattern, in particular, the 10,11-dihydroxy substituents. However, the 10,11-dioxy substituents need not be in the form of hydroxyl groups. Masked hydroxyl groups, or prodrug (hydroxyl protecting) groups can also be used. For example, esterification of the 10,11-hydroxyl groups with, for example, benzoic acid or pivalic acid ester forming compounds (e.g., acid anhydrides) yields 10,11-dibenzoyl or dipivaloyl esters that are useful as prodrugs, i.e., they will be hydrolyzed in vivo to produce the biologically active 10,11-dihydroxy compound. A variety of biologically acceptable carboxylic acids can also be used. Furthermore, the 10,11-dioxy ring substitution can be in the form of a 10,11-methylenedioxy or ethylenedioxy group. In vivo, body metabolism will cleave this linkage to provide the more active 10,11-dihydroxy functionality. Compound potency and receptor selectivity can also be affected by the nature of the nitrogen substituent.

In another embodiment of the method and composition described herein, C₂, C₃, and/or C₄-substituted trans-5,6,6a,7,8,12b-hexahydrobenzo[α]phenanthridines can be used as the D₁ dopamine receptor agonist. The selectivity of these compounds for dopamine receptor subtypes varies, depending on the nature and positioning of substituent groups. Substitution at the C₂, C₃, and/or C₄ position on the benzophenanthridine ring system controls affinity for the dopamine receptor subtypes and concomitantly receptor selectivity. Thus, for example, 2-methyldihydrexidine has D₁ potency and efficacy comparable to dihydrexidine, while it has a five-fold enhanced selectivity for the D₁ receptor. In contrast, the compound 3-methyldihydrexidine, although retaining D₁ potency and efficacy comparable to dihydrexidine, has greater D₂ potency, making it less selective but better able to activate both types of receptors.

In another embodiment of compounds of formula II, where X is —OR⁵, R¹ and R⁵ are different. In one aspect, one of R¹ and R⁵ is hydrogen or acetyl and the other of R¹ and R⁵ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms, and the physiologically acceptable salts thereof.

In another embodiment, chromeno[4,3,2-de]isoquinoline compounds can be used as the D₁ dopamine receptor agonist administered in combination therapy with a D₂ dopamine receptor antagonist. Exemplary compounds that are used in the method and composition described herein include, but are not limited to compounds having Formula III: 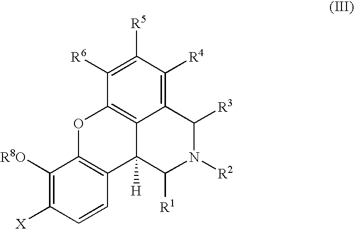
wherein R¹, R², and R³ are each independently selected from hydrogen, C₁-C₄ alkyl, and C₂-C₄ alkenyl, R⁸ is hydrogen, C₁-C₄ alkyl, acyl, an active ester group, or an optionally substituted phenyl protecting group, X is hydrogen, halo including fluoro, chloro, bromo, and iodo, or a group of the formula —OR⁹ wherein R⁹ is hydrogen, C₁-C₄ alkyl, acyl, or an optionally substituted phenoxy protecting group, and R⁴, R⁵, and R⁶ are each independently selected from the group consisting of hydrogen, C₁-C₄ alkyl, phenyl, halo, and a group —OR wherein R is hydrogen, acyl, such as benzoyl, pivaloyl, and the like, or an optionally substituted phenyl protecting group, and when X is a group of the formula —OR⁹, the groups R⁸ and R⁹ can be taken together to form a group of the formula —CH₂— or —(CH₂)₂—. The compounds also comprise pharmaceutically acceptable salts thereof.

It is appreciated that compounds having Formula III are chiral. It is further appreciated that although a single enantiomer is depicted, each enantiomer alone and/or various mixtures of each enantiomer, including racemic mixtures, are contemplated, and may be included in the compounds, compositions, and methods described herein.

In this embodiment, “C₂-C₄ alkenyl” as used herein refers to branched or straight-chain alkenyl groups having two to four carbons, such as allyl, 2-butenyl, 3-butenyl, and vinyl.

In another embodiment, wherein compounds of Formula III are used in the method and composition described herein at least one of R₄, R₅, or R₆ is hydrogen. In another embodiment at least two of R₄, R₅, or R₆ are hydrogen.

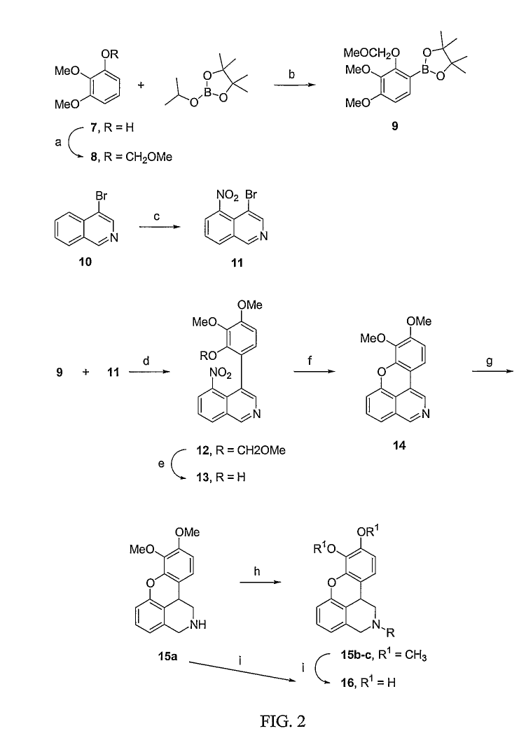

One compound of Formula III for use in the method and composition described herein is (±)-8,9-dihydroxy-1,2,3,11b-tetrahydrochromeno[4,3,2-de]isoquinoline hydrobromide (16a), denominated hereinafter as “dinoxyline.” Dinoxyline is synthesized from 2,3-dimethoxyphenol (7) and 4-bromoisoquinole (10), as depicted in FIG. 2. The phenolic group is protected as the methoxymethyl (“MOM”) derivative 8 followed by treatment with butyllithium, then with the substituted borolane illustrated, to afford the borolane derivative 9.

As shown in FIG. 2, this borolane derivative is then employed in a Pd-catalyzed Suzuki type cross coupling reaction with 5-nitro-4-bromoisoquinoline (11), prepared from bromoisoquinoline 10. The resulting coupling product 12 is then treated with toluenesulfonic acid in methanol to remove the MOM protecting group of the phenol. Treatment of this nitrophenol 13 with potassium carbonate in DMF at 80° C. leads to ring closure with loss of the nitro group, affording the basic tetracyclic chromenoisoquinoline nucleus 14. Catalytic hydrogenation effects reduction of the nitrogen-containing ring to yield 15a. Use of boron tribromide to cleave the methyl ether linkages gives the parent compound 16a.

It is apparent that by appropriate substitution on the isoquinoline ring a wide variety of substituted compounds can be obtained. Substitution onto the nitrogen atom in either 14 or 15a, followed by reduction will readily afford a series of compounds substituted with lower alkyl groups on the nitrogen atom. Likewise, the use of alkyl substituents on the 1, 3, 6, 7, or 8 positions of the nitroisoquinoline 11 leads to a variety of ring-substituted compounds. In addition, the 3-position of 14 can also be directly substituted with a variety of alkyl groups. Similarly, replacement of the 4-methoxy group of 9, in FIG. 2, with fluoro, chloro, or alkyl groups leads to the subject compounds with variations at X₉. When groups are present on the nucleus that are not stable to the catalytic hydrogenation conditions used to convert 14 to 15a, reduction can be accomplished using sodium cyanoborohydride at slightly acidic pH. Further, formation of the N-alkyl quaternary salts of derivatives of 14 gives compounds that are also easily reduced with sodium borohydride, leading to derivatives of 15a.

FIG. 2 also illustrates the synthesis of N-substituted chromenoisoquinolines 15 and 16. Compound 15a is N-alkylated under standard conditions to provide substituted derivatives. Alkylating agents, such as R-L, where R is methyl, ethyl, propyl, allyl, and the like, and L is a suitable leaving group such as halogen, methylsulfate, or a sulfonic acid derivative, are used to provide the corresponding N-alkyl derivatives. The aromatic methyl ethers of compounds 15 are then removed under standard conditions, such as upon treatment with BBr₃ and the like. It appreciated that N-alkylation may be followed by other chemical transformations to provide the substituted derivatives described herein. For example, alkylation with an allyl halide followed by hydrogenation of the allyl double bond provides the corresponding N-propyl derivative.

In another embodiment of compounds of formula III, where X is —OR⁹, R⁸ and R⁹ are different. In one aspect, one of R⁸ and R⁹ is hydrogen or acetyl and the other of R⁸ and R⁹ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)allyl, and (C₁-C₃)alkoxy, which latter may in turn be substituted by 1 to 3 halogen atoms, and the physiologically acceptable salts thereof.

In another embodiment, tetrahydronaphtho[1,2,3-de]isoquinoline compounds are used as the D₁ dopamine receptor agonist for co-administration with a D₂ dopamine receptor antagonist. Exemplary compounds for use in the method and composition described herein include, but are not limited to compounds having Formula IV: 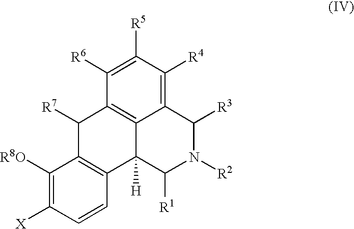
and pharmaceutically acceptable salts thereof, wherein R¹, R², and R³ are each independently selected from the group consisting of hydrogen, C₁-C₄ alkyl, and C₂-C₄ alkenyl; R⁴, R⁵, and R⁶ are each independently selected from the group consisting of hydrogen, C₁-C₄ alkyl, phenyl, halogen, and a group having the formula —OR, where R is hydrogen, acyl, an active ester group, or an optionally substituted phenyl protecting group; R⁷ is selected from the group consisting of hydrogen, hydroxy, C₁-C₄ alkyl, C₂-C₄ alkenyl, C₁-C₄ alkoxy, and C₁-C₄ alkylthio; R⁸ is hydrogen, C₁-C₄ alkyl, acyl, or an optionally substituted phenyl protecting group; and X is hydrogen, fluoro, chloro, bromo, or iodo, or a group —OR⁹ wherein R⁹ is hydrogen, benzoyl, pivaloyl, an active ester group, or an optionally substituted phenyl protecting group; or when X is the group —OR⁹, R⁸ and R⁹ are taken together to form a divalent radical selected from the group consisting of —CH₂— and —(CH₂)₂—.

It is appreciated that compounds having Formula IV are chiral. It is further appreciated that although a single enantiomer is depicted, each enantiomer alone and/or various mixtures of each enantiomer, including racemic mixtures, are contemplated, and may be included in the compounds, compositions, and methods described herein.

In another embodiment of Formula IV, X is a group having the formula —OR⁹, where R⁹ is hydrogen, C₁-C₄ alkyl, acyl, or an optionally substituted phenyl protecting group; or the groups R⁸ and R⁹ are taken together to form a divalent group having the formula —CH₂— or —(CH₂)₂—.

In accordance with the method and composition described herein, the term “pharmaceutically acceptable salts” as used herein refers to those salts formed using organic or inorganic acids that are suitable for use in contact with the tissues of humans and lower animals without undue toxicity, irritation, allergic response, and the like. Acids suitable for forming pharmaceutically acceptable salts of biologically active compounds having amine functionality are well known in the art. The salts can be prepared according to conventional methods in situ during the final isolation and purification of the present compounds, or separately by reacting the isolated compounds in free base form with a suitable salt forming acid.

In accordance with the method and composition described herein, the term “phenoxy protecting group” as used herein refers to substituents on the phenolic oxygen which prevent undesired reactions and degradations during synthesis and which can be removed later without effect on other functional groups on the molecule. Such protecting groups and the methods for their application and removal are well known in the art. They include ethers, such as methyl, isopropyl, t-butyl, cyclopropylmethyl, cyclohexyl, allyl ethers and the like; alkoxyalkyl ethers such as methoxymethyl or methoxyethoxymethyl ethers and the like; alkylthioalkyl ethers such a methylthiomethyl ethers; tetrahydropyranyl ethers; arylalkyl ethers such as benzyl, o-nitrobenzyl, p-methoxybenzyl, 9-anthrylmethyl, 4-picolyl ethers and the like; trialkylsilyl ethers such as trimethylsilyl, triethylsilyl, t-butyldimethylsilyl, t-butyldiphenylsilyl ethers and the like; alkyl and aryl esters such as acetates, propionates, n-butyrates, isobutyrates, trimethylacetates, benzoates and the like; carbonates such as methyl, ethyl, 2,2,2-trichloroethyl, 2-trimethylsilylethyl, vinyl, benzyl and the like; and carbamates such as methyl, isobutyl, phenyl, benzyl, dimethyl and the like.

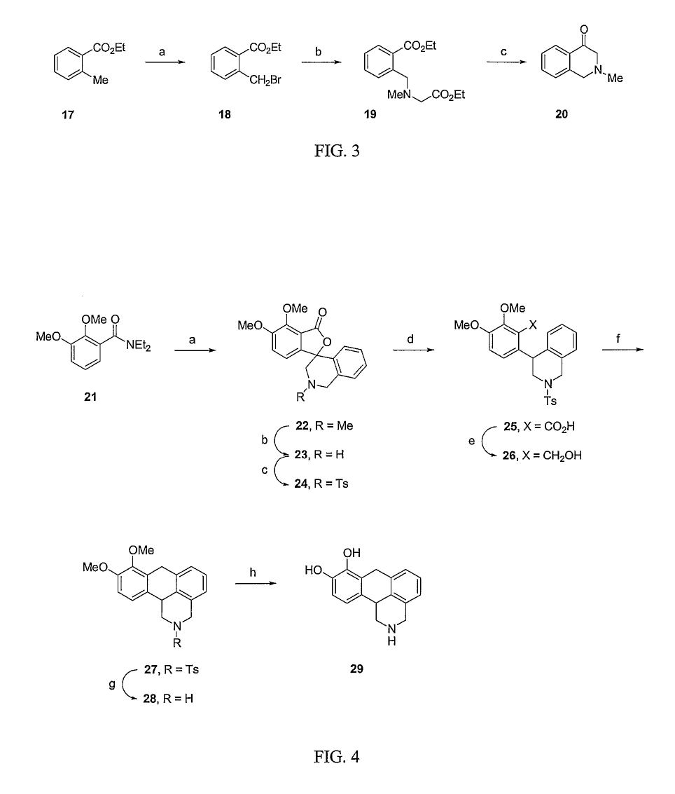

One compound for use in accordance with the method and composition described herein as a D₁ dopamine receptor agonist for co-administration with a D₂ dopamine receptor antagonist is (±)-8,9-dihydroxy-2,3,7,11b-tetrahydro-1H-naphtho-[1,2,3-de]-isoquinoline (29) denominated hereinafter as “dinapsoline.” Dinapsoline is synthesized from 2-methyl-2,3-dihydro-4(1H)-isoquinolone (20) according to the procedure depicted generally in FIGS. 3 and 4. Side chain bromination of ethyl 2-toluate (17) with NBS in the presence of benzoyl peroxide produced compound 18. Alkylation of sarcosine ethyl ester with compound 18 afforded compound 19, which after Dieckmann condensation and subsequent decarboxylation on acidic hydrolysis yielded compound 20.

As shown in FIG. 4, ortho-directed lithiation of 2,3-dimethoxy-N,N′-diethylbenzamide (21) with sec-butyllithium/TMEDA in ether at −78° C. and condensation of the lithiated species with compound 20 followed by treatment with p-toluene sulfonic acid at reflux gave spirolactone 22 in modest yield. N-Demethylation of 22 with 1-chloroethylchloroformate followed by methanolysis of the intermediate afforded compound 23, that on treatment with p-toluenesulfonyl chloride and triethylamine provided compound 24.

Early attempts to synthesize compound 24 directly by condensation of 2-p-toluenesulfonyl-2,3-dihydro-4(1H)-isoquinolone with lithiated compound 21 in THF or ether, followed by lactonization with acid provided only trace amounts (<5%) of compound 24. Enolization of 2-p-toluenesulfonyl-2,3-dihydro-4(1H)-isoquinolone under the basic reaction conditions is one possible explanation for the poor yield.

Hydrogenolysis of compound 24 in glacial acetic acid in the presence of 10% palladium on carbon gave compound 25 that on reduction with diborane afforded intermediate compound 26. Cyclization of compound 26 with concentrated sulfuric acid at low temperature provided compound 22. N-Detosylation of compound 22 with Na/Hg in methanol buffered with disodium hydrogen phosphate gave compound 28. Finally, compound 28 was treated with boron tribromide to effect methyl ether cleavage yielding dinapsoline (29) as its hydrobromide salt.

Alternatively, dinapsoline and compounds related to dinapsoline may also be synthesized according to the procedure described by Sattelkau, Qandil, and Nichols, “An efficient synthesis of the potent dopamine D₁ agonst dinapsoline by construction and selective reduction of 2′-azadimethoxybenzanthrone,” Synthesis 2:262-66 (2001), the entirety of the description of which is incorporated herein by reference.

In another embodiment of compounds of formula IV, where X is —OR⁹, R⁸ and R⁹ are different. In one aspect, one of R⁸ and R⁹ is hydrogen or acetyl and the other of R⁸ and R⁹ is selected from the group consisting of (C₃-C₂₀)alkanoyl, halo-(C₃-C₂₀)alkanoyl, (C₃-C₂₀)alkenoyl, (C₄-C₇)cycloalkanoyl, (C₃-C₆)-cycloalkyl(C₂-C₁₆)alkanoyl, aroyl which is unsubstituted or substituted by 1 to 3 substituents selected from the group consisting of halogen, cyano, trifluoromethanesulphonyloxy, (C₁-C₃)alkyl and (C₁-C₃)alkoxy, which latter may in turn be substituted by 1 to 3 halogen atoms, aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, which latter may in turn be substituted by 1 to 3 halogen atoms: and hetero-arylalkanoyl having one to three heteroatoms selected from O, S and N in the heteroaryl moiety and 2 to 10 carbon atoms in the alkanoyl moiety and which is unsubstituted or substituted in the heteroaryl moiety by 1 to 3 substituents selected from the group consisting of halogen, cyano, trifluoromethanesulphonyloxy, (C₁-C₃)alkyl, and (C₁-C₃)alkoxy, which latter may in turn be substituted by 1 to 3 halogen atoms, and the physiologically acceptable salts thereof.

In an alternative embodiment, compounds 35 may be prepared from optionally substituted isoquinolines 30, which generally undergo electrophilic substitution preferentially at the 5-position to give 5-bromo-isoquinolines 31. The bromination reaction is illustratively performed neat in the presence of a Lewis Acid catalyst, such as anhydrous aluminum chloride, or alternatively in an inert organic solvent, such as methylene chloride. 5-bromo-isoquinolines 31 can be trans-metallated to the corresponding 5-lithio-isoquinolines using n-butyl lithium in a suitable inert organic solvent such as THF, illustratively at a temperature less than about −50, or about −80° C., followed by alkylation, or optionally acylation, to form the corresponding 5-substituted isoquinolines. Acylation with DMF gives, followed by warming to room temperature and neutralization with an equivalent amount of mineral acid, gives 5-formyl-isoquinolines 32. Aldehyde 32 is reacted with 4-bromo-3-lithio-1,2-(methylenedioxy)benzene 34, prepared by conventional ortho-lithiation methods from the corresponding substituted benzene 33, to give 35.

Cyclization of 35 to the corresponding compounds 36 can be initiated by free radical initiated carbon-carbon bond formation, or by a variety of conventional reaction conditions. The carbon-carbon bond reaction is illustratively carried out with a hydrogen radical source such as trialkyltin hydride, triaryltin hydride, trialkylsilane, triarylsilane, and the like, and a radical initiator, such as 2,2′-azobisisobutylronitrile, sunlight, UV light, controlled potential cathodic (Pt), and the like in the presence of a proton source such as a mineral acid, such as sulfuric acid, hydrochloric acid, and the like, or an organic acid, such as acetic acid, trifluoroacetic acid, p-toluenesulfonic acid, and the like. Illustratively, 36 is prepared by treatment with tributyltin hydride and, 2,2′-azobisisobutylronitrile in the presence of acetic acid.

Compounds 36 are selectively reduced at the nitrogen bearing heterocyclic ring to give the corresponding tetrahydroisoquinolines 37. The selective ring reduction may be carried out by a number of different reduction methods such as sodium cyanoborohydride in an acidic medium in THF, hydride reducing agents such as L-SELECTRIDE or SUPERHYDRIDE, catalytic hydrogenation under elevated pressure, and the like. Conversion of the protected compounds 37 to diols 38 may be accomplished using boron tribromide in methylene chloride at low temperatures, such as less than about −60, or less than about −80° C. Compounds 38 may be isolated as the hydrobromide salt. The corresponding hydrochloride salt may also be prepared by using boron trichloride.

In another embodiment of compounds of formula IV, an optically active preparation is described. The substantially pure (+)-isomer and (−)-isomer of compounds 38 are prepared by chiral separation of the hydroxy-protected compounds 37, by forming a chiral salt, such as the (+)-dibenzoyl-D-tartaric acid salt of compounds 37, followed by removal of the protecting group as described herein. It is appreciated that other racemic mixtures of compounds described herein, such as compounds of formulae I, II, III, V, and VI, may also be separated by resolution of optically active salts, or by chiral column chromatography. Alternative procedures are described in Knoerzer et al. (1994) “Dopaminergic benzo[a]phenanthridines: resolution and pharmacological evaluation of the enantiomers of dihydrexidine, the full efficacy D₁ dopamine receptor agonist” J. Med. Chem., 37:2453-2460, the description of the synthesic preparations of which are incorporated herein by reference.

In another embodiment, heterocyclic-fused phenanthridine compounds, such as thieno[1,2-α]phenanthridines, and the like, are used as the D₁ dopamine receptor agonist for administration in combination therapy with a D₂ dopamine receptor antagonist to patients with neurological disorders. Exemplary compounds for use in the methods and compositions described herein include, but are not limited to, compounds having Formula V:

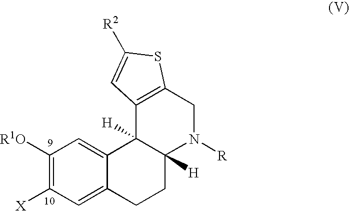

and pharmaceutically acceptable salts thereof; R is hydrogen or C₁-C₄ alkyl; R¹ is hydrogen, acyl, such as C₁-C₄ alkanoyl, benzoyl, pivaloyl, and the like, or a phenoxy protecting group; X is hydrogen, fluoro, chloro, bromo, iodo, or a group of the formula —OR³ wherein R³ is hydrogen, alkyl, acyl, or a phenoxy protecting group, provided that when X is a group of the formula —OR³, the groups R¹ and R³ can be taken together to form a —CH₂- group or a —(CH₂)₂— group, thus representing a methylenedioxy or ethylenedioxy functional group bridging the C-9 and C-10 positions; and R² is selected from the group consisting of hydrogen, C₁-C₄ alkyl, phenyl, fluoro, chloro, bromo, iodo, or a group —OR⁴ wherein R⁴ is hydrogen, alkyl, acyl, or a phenoxy protecting group.

It is appreciated that compounds having Formula V are chiral. It is further appreciated that although a single enantiomer is depicted, each enantiomer alone and/or various mixtures of each enantiomer, including racemic mixtures, are contemplated, and may be included in the compounds, compositions, and methods described herein.

Exemplary compounds of Formula V include, but are not limited to, ABT 431 (X=CH₃CO₂, R¹=CH₃CO, R²=CH₃(CH₂)₂, R=H) and A 86929 (X=OH, R¹=H, R=CH₃(CH₂)₂, R=H).

In another embodiment, phenyltetrahydrobenzazepine compounds can be used as the D₁ dopamine receptor agonist for co-administration with a D₂ dopamine receptor antagonist. Exemplary compounds for use in the method and composition described herein include, but are not limited to compounds having Formula VI: 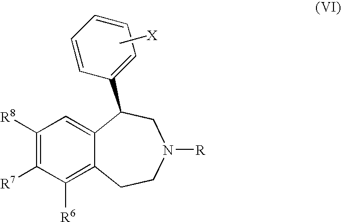
wherein R is hydrogen, alkyl, alkenyl, optionally substituted benzyl, or optionally substituted benzoyl; R6, R7, and R8 are each independently selected from hydrogen, halogen, hydroxy, alkyl, alkoxy, and acyloxy; and X is hydrogen, halogen, hydroxy, alkyl, alkoxy, or acyloxy. Illustrative compounds having the Formula VI include SKF 38393 (R6=H, R7=R8=OH, R=H, X=H), SKF 82958 (R6=Cl, R7=R8=OH, R=CH2CH=CH2, X=H), SKF 81297 (R6=Cl, R7=R8=OH, R=H, X=H, and described in Eur. J. Pharmacol. 188:335 (1990)), and SCH 23390 (R6=H, R7=Cl, R8=OH, R=CH3, X=H).

It is appreciated that compounds having Formula VI are chiral. It is further appreciated that although a single enantiomer is depicted, each enantiomer alone and/or various mixtures of each enantiomer, including racemic mixtures, are contemplated, and may be included in the compounds, compositions, and methods described herein.

It is to be understood that other D₁ receptor agonists may be included in the compounds, compositions, and methods described herein, including but not limited to A68930 ((1R,3 S)-l-aminomethyl-5,6-dihydroxy-3-phenylisochroman hydrochloride), A77636 ((1R,3S)-3-(1′-adamantyl)-1-aminomethyl-3,4-dihydro-5,6-dihydroxy-1H-2-benzopyran), and the like. A77636 may be prepared according to DeNinno et al., Eur. J. Pharmacol. 199:209-19 (1991) and/or DeNinno et al., J. Med. Chem. 34:2561-69 (1991), the disclosures of which are incorporated herein by reference.

In another embodiment, the dopamine D₁ receptor agonist is selected based on a predetermined half-life. Illustratively, dihydrexidine has a short-half life of about 30 min when given intravenously, and a functional half-life of about 3 hr when given subcutaneously. In contrast, dinapsoline has a 3 hr serum half-life with about 7-10 hr of functional activity.

As used herein, the term “effective amounts” refers to amounts of the compounds which prevent, reduce, or stabilize one or more of the clinical symptoms of disease in a patient at risk of developing or suffering from pulmonary edema, or a disease that results in pulmonary edema. It is appreciated that the effective amount may improve the condition of a patient either permanently or temporarily. The clearance of pulmonary edema may prevent subsequent hypoxia, shock, and death in patients; decrease or prevent secondary bacterial infections caused by bacterial overgrowth in the rich edema fluid; and permit the access of conventional drugs for treatment, such as effective antimicrobials to the target virus.

It is generally accepted that there are at least two pharmacological subtypes of dopamine receptors (the D₁ and D₂ receptor subtypes), each consisting of several molecular forms. Dopamine D₁ receptors preferentially recognize the phenyltetrahydrobenzazepines and generally lead to stimulation of the enzyme adenylate cyclase, whereas dopamine D₂ receptors recognize the butyrophenones and benzamides and often are coupled negatively to adenylate cyclase, or are not coupled at all to this enzyme. It is now known that at least five dopamine receptor genes encode the dopamine D₁, D₂, D₃, D₄ and D₅ receptor isoforms or subtypes. The traditional classification of dopamine receptor subtypes, however, remains useful with the dopamine D₁-like class comprising the D₁ (D_{1A} in rat) and the D₅ (D_{1B} in rat) receptor subtypes, whereas the dopamine D₂-like class consists of the D₂, D₃ and D₄ receptor subtypes. As the effects caused by association of selective ligands with specific receptor subtypes become better understood, drug researchers will be much better positioned to design drugs targeting specific disease states or disorders. The biological activities of the compounds useful in the methods described herein range from compounds that are either full agonists or partial agonists at dopamine D₁ receptors, that are selective for or more active at dopamine D₁ receptors than other monoamine receptors, and that are selective for or more active at dopamine D₁ receptors than dopamine D₂ receptors.

In one illustrative embodiment, the compounds described herein include full dopamine D₁ receptor agonists. In another illustrative embodiment, the compounds described herein include selective dopamine D₁ receptor agonists that are more active at dopamine D₁ receptors than at one or more other monoamine receptors, including but not limited to, adrenergic receptors, serotonergic receptors, and the like. In another illustrative embodiment, the compounds described herein include selective dopamine D₁ receptor agonists that are more active at dopamine D₁ receptors than other dopamine receptors.

In one aspect, dihydrexidine; (DHX, (±)-trans-10,11-dihydroxy-5,6,6a,7,8,12b-hexahydrobenzo[a]phenanthridine) is described. DHX is a potent, full-efficacy dopamine D₁ receptor agonist that binds with high affinity to the dopamine D₁ receptor with an IC₅₀ of about 10 nM. DHX is inactive (IC50>10 uM) at about 40 different receptor binding sites, and shows lower activity at dopamine D₂ receptors (IC₅₀ =130 uM) and adrenergic receptors (IC₅₀=230 nM). See, Mottola et al. (1992) “Dihydrexidine, a novel full efficacy D1 dopamine receptor agonist” J. Pharmacol. Exp. Ther., 262:383-393. DHX is as efficacious as dopamine and approximately 70 times more potent in the stimulation of adenylate cyclase. This effect is blocked by the dopamine D₁ antagonist SCH 23390, but not by dopamine D₂, 5-HT₂, muscarinic, or adrenergic receptor antagonists. Despite its D₂ affinity, the functional effects of DHX, both in situ and in vivo, are attributable almost entirely to the activity at dopamine D₁ receptors. This observation is believed to be attributable to the functional selectivity of the compound at the D₂ receptor. Further, binding and functional activity are associated primarily with the trans-dextrorotatory enantiomer. DHX has shown efficacy in causing fluid absorption from injured rat lung after intravenous administration.

Conventional dopamine receptor agonists have low affinity for the dopamine D₁ receptor and/or low selectivity compared to either other dopamine receptor subtypes, or other monoamine receptors. Further, many conventional dopamine receptor are only partial agonists. For example, dopamine (Intropin) has relatively low affinity for the dopamine D₁ receptor of about 500 nM, but will also activate other dopamine, adrenergic, and even serotonergic receptors when the dose is increased. Further, dobutamine (Dobutrex), a dopamine receptor agonist, has higher affinity for adrenoreceptors than for dopamine receptors. Further, fenoldopam, a dopamine receptor agonist, is only slightly more potent that dopamine, has a very short half-life of about 15 minutes, and can be used only for up to 48 hours due to the development of tolerance. Fenoldopam has been shown to increase smooth muscle tension, an effect that is not blocked by the dopamine D₁ antagonist SCH23390, but is blocked by the serotonin antagonists ketanserin and methylsergide.

It is appreciated that side effects seen with compounds that do not exhibit selectivity for dopamine receptors over other monoamine receptors, or that do not exhibit selectivity for dopamine D₁ receptors over dopamine D₂ receptors may be avoided by embodiments of the compounds described herein. Such side effects may include increased risk of myocardial infarction, congestive heart failure, cardiac arrest, sudden cardiac death, seizures, hypotension, increased intraocular pressure, mild appetite suppression, headache, nausea, emesis, hallucinations, confusion, psychosis, and sleep disturbances.

In another embodiment, the systemic absorption of the dopamine receptor agonist from the endobronchial space is insubstantial. In alternative embodiments of the present method less than about 1%, less than about 3%, less than about 5%, less than about 7%, less than about 10%, less than about 12%, less than about 15%, less than about 17%, or less than about 20% of the dopamine receptor agonist administered to the patient is absorbed systemically.

As used herein, the term “D₁ receptor” refers to each and every D₁ and D₁-like receptor, alone or in various combinations, including the D₁ and D₅ receptors in humans, the D_{1A} and D_{1B} receptors found in rats, and other D₁-like receptors.

It is appreciated that in certain variations of the compounds, compositions, and methods described herein, full dopamine D₁ agonists are included and partial dopamine D₁ agonists are excluded. For certain diseases states, or disease stages, partial dopamine D₁ agonists may not be as effective as full dopamine D₁ agonists. Illustrative of this variation, compounds of formulae I-IV are used in the compounds, compositions, and methods described herein, and in particular those examples of formulae I-IV that are full dopamine D₁ receptor agonists.

It is to be further understood that references to receptor selectivity include functional selectivity at dopamine receptors. Such functional selectivity may further distinguish the activity of the compounds and compositions described herein to allow the treatment of more specifically predetermined symptoms. For example, compounds and compositions that are selective for a particular dopamine receptor, illustratively the D₁ receptor, may yet exhibit a second layer of selectivity where such compounds and compositions show functional activity at dopamine D₁ receptors in one or more tissues, but not in other tissues. Illustrative of such functional selectivity is the reported selectivity of dihydrexidine for postsynaptic neurons over presynaptic neurons. Other functional selectivity is contemplated herein.

Dopamine D₁ receptor agonists, including dihydrexidine (DHX), have been shown to be physiologically linked to clinically relevant targets in the treatment of pulmonary edema. DHX is a full D1 agonist that includes two chiral centers, generating four possible diasteromers. The majority of the dopamine D₁ receptor agonist activity lies in the trans (+) enantiomer. DHX has been evaluated in rabbit and rat models of lung injury and shown to induce fluid resorption when administered by an intravenous route. DHX is a high potency, full D₁ agonist that is poorly absorbed after oral administration and has a short serum half-life (less than 10 min when given intravenously); making it ideal for pulmonary administration to the target organ and has minimum potential for side effects as it is metabolized rapidly even if it is absorbed systemically.

DHX exhibits relatively poor oral bioavailability and a short in vivo half-life. Those properties make DHX favorable as an adjunct therapeutic for treating pulmonary edema, secondary to viral infections. The desirable characteristic of DHX for pulmonary delivery are low passage through cellular barriers.

The compounds for use in the methods described herein may be formulated in conventional drug dosage forms for endobronchial administration, such as in an aerosol form or in the form of a dry powder. Illustrative doses of the compounds for use in the methods depend on many factors, including the indication being treated and the overall condition of the patient. For example, in one embodiment effective amounts of the present compounds range from about 1.0 ng/kg to about 15 mg/kg of body weight. In another embodiment, effective amounts range from about 50 ng/kg to about 10 mg/kg of body weight. In another embodiment, effective amounts range from about 200 ng/kg to about 5 mg/kg of body weight. In another embodiment, effective amounts range from about 300 ng/kg to about 3 mg/kg of body weight. In another embodiment, effective amounts range from about 500 ng/kg to about 1 mg/kg of body weight. In another embodiment, effective amounts range from about 1 μg/kg to about 0.5 mg/kg of body weight. In general, treatment regimens utilizing compounds in accordance with the present invention comprise administration of from about 10 ng to about 100 mg of the compounds for use in the method of this invention per day in multiple doses or in a single dose. Effective amounts of the compounds for use in the method of the invention can be administered using any regimen such as once daily or twice daily. In one aspect, the treatment regimen is maintained for at least one day to about twenty-one days.

Illustratively, the active compound is admixed with an inert diluent or carrier appropriate for delivery of the compound to the lung endobronchial space of the airways of the patient. The diluent can be any conventional physiologically acceptable diluent, such as water or saline, for example. The compositions for administration to a patient can also contain adjuvants, such as wetting agents, and emulsifying and suspending agents. The dosage forms of the compounds for use in the method of the present invention can be formulated using art-recognized techniques for formulating aerosols and dry powders for delivery to the lungs, and can be sterilized using conventional microfiltration techniques.

In accordance with one embodiment, a pharmaceutical composition is provided comprising effective amounts of the active ingredients, and a pharmaceutically acceptable carrier therefor. A “pharmaceutically acceptable carrier” for use in accordance with the method and composition described herein is compatible with other reagents in the pharmaceutical composition and is not deleterious to the patient.

### EXAMPLES

The following examples are illustrative of the compounds for use in the methods and compositions described herein, and are not intended to limit the invention to the disclosed compounds. Other compounds that can be used in accordance with the claimed method include those compounds described in U.S. Pat. Nos. 5,047,536, 5,420,134, 5,959,110, 6,413,977, and 6,147,072. Each of these patents is incorporated herein by reference. Obvious variations and modifications of the exemplified compounds are also intended to be within the scope of the compounds, compositions, and methods described herein. The following examples are also illustrative of the formulations and delivery systems useful in the methods described herein, and are not intended to limit the invention in any way.

With reference to the experimental procedures described herein, unless otherwise indicated, the following procedures were used where applicable. Solvent removal was accomplished by rotary evaporation under reduced pressure. Melting points were determined with a Thomas-Hoover melting point apparatus and are uncorrected. ¹H NMR spectra chemical shifts are reported in values (ppm) relative to TMS. The IR spectra were recorded as KBr pellets or as a liquid film. Mass spectra were obtained using chemical ionization (CIMS). When anhydrous conditions were required, THF was distilled from benzophenone-sodium ketyl under N₂ immediately before use, and 1,2-Dichloroethane was distilled from phosphorous pentoxide before use.

### Example 1

### Dihydrexidine (6a)

2-(N-Benzyl-N-benzoyl)-6,7-dimethoxy-3,4-dihydro-2-napthylamine (2a). To a solution of 4.50 g (21.8 mmol) of 6,7-dimethoxy-β-tetralone (1) in 100 mL of toluene was added 2.46 g (23 mmol) of benzylamine. The reaction was heated at reflux overnight under N₂ with continuous water removal. The reaction was cooled, and the solvent was removed to yield N-benzyl enamine as a brown oil.

This residue was dissolved in 80 mL of CH₂Cl₂, and to this was added 2.43 g (24 mmol) of triethylamine, and the solution was cooled in an ice bath. Benzoyl chloride (3.37 g, 24 mmol) was then dissolved in 15 mL of CH₂Cl₂ and this solution was then added dropwise to the cold stirring N-benzyl enamine solution. After complete addition the reaction was allowed to warm to room temperature and was left to stir overnight. The mixture was then washed successively with 2×50 mL of 5% aqueous HCl, 2×50 mL of 1 N NaOH, saturated NaCl solution, and was then dried over MgSO₄. After filtration, the filtrate was concentrated. Crystallization from diethyl ether gave 5.6 g (64%) of enamide 2: mp 109-110° C.; IR (KBr) 1620 cm⁻¹; CIMS (isobutane, M+1) 400; ¹H-NMR (CDCl₃) δ 7.64 (m, 2, ArH), 7.33 (m, 8, ArH), 6.52 (s, 1, ArH), 6.38 (s, 1, ArH), 6.05 (s, 1, ArCH), 4.98 (s, 2, ArCH₂ N), 3.80 (s, 3, OCH₃), 3.78 (s, 3, OCH₃), 2.47 (t, 2, CH₂, J=8.1 Hz), 2.11 (t, 2, CH₂, J=8.1 Hz).

Trans-6-benzyl-10,1 1-dimethoxy-5,6,6a,7,8,12b-hexahydrobenzo[α]phenanithridine-5-one (3a). A solution of 3.14 g (7.85 mmol) of the 6,7-dimethoxyenamide 2, in 300 mL of THF, was introduced into an Ace Glass 250 mL photochemical reactor. This solution was stirred while irradiating for 5 hours with a 450 watt Hanovia medium pressure, quartz, mercury-vapor lamp seated in a water cooled, quartz immersion well. The solution was concentrated and crystallized from ether to provide 1.345 g (42.9%) of 3a: mp 183-186° C.; IR (KBr) 1655, 1640 cm⁻¹; CIMS (isobutane, M+1) 400; ¹H-NMR (CDCl₃) δ 8.19 (m, 1 ArH), 7.52 (m, 1, ArH), 7.46 (m, 2, ArH), 7.26 (m, 5, ArH), 6.92 (s, 1, ArH), 6.63 (s, 1, ArH), 5.35 (d, 1, ArCH₂N, J=16.0 Hz), 4.78 (d, 1, ArCH₂ N, J=16.0 Hz), 4.37 (d, 1, Ar₂CH, J=11.3 Hz), 3.89 (s, 3, OCH₃), 3.88 (s, 3, OCH₃), 3.80 (m, 1 CHN), 2.67 (m, 2, ArCH₂), 2.25 (m, 1, CH₂CN), 1.75 (m, 1, CH₂CN).

Trans-6-benzyl-10,11-dimethoxy-5,6,6a,7,8,12b-hexahydrobenzo[α]phenanthridine hydrochloride (4a). A solution of 1.20 g (3 mmol) of 3a, in 100 mL of dry THF was cooled in an ice-salt bath and 6.0 mL of 1 M BH₃ was added via syringe. The reaction was heated at reflux overnight. Water (10 mL) was added dropwise, and the reaction mixture was concentrated by distillation at atmospheric pressure. The residue was stirred with 50 mL of toluene, 1.0 mL of methane sulfonic acid was added, and the mixture was heated with stirring on the steam bath for one hour. The mixture was diluted with 40 mL of water and the aqueous layer was separated. The toluene was extracted several times with water, and the aqueous layers were combined. After basification of the aqueous phase with conc. ammonium hydroxide, the free base was extracted into 5×25 mL of CH₂Cl₂. This organic extract was washed with saturated NaCl solution, and dried over MgSO₄. After filtration, the organic solution was concentrated, the residue was taken up into ethanol, and carefully acidified with concentrated HCl. After drying several times by azeotropic distillation of ethanol, crystallization from ethanol afforded 0.97 g (76.5%) of the salt 4a: mp 235-237° C.; CIMS (NH₃, M+1) 386; ¹H-NMR (CDCl₃, free base) δ 7.37 (m, 9 ArH), 6.89 (s, 1, ArH), 6.74 (s, 1, ArH), 4.07 (d, 1, Ar₂CH, J=10.7 (Hz), 3.90 (s, 3, OCH₃), 3.82 (m, 2, ArCH₂N), 3.79 (s, 3, OCH₃), 3.52 (d, 1 ArCH₂N, J=15.3 Hz), 3.30 (d, 1, ArCH₂), J=13.1 Hz), 2.86 (m, 2, CHN, ArCH₂), 2.30 (m, 2, ArCH₂, CH₂CN), 1.95 (m, 1, CH₂CN).

Trans-10,11-dimethoxy-5,6,6a,7,8,12b-hexahydrobenzo[α]phenanthridine hydrochloride (5a). A solution of 0.201 g (0.48 mmol) of the 6-benzyl hydrochloride salt 4a in 50 mL of 95% ethanol containing 50 mg of 10% Pd-C catalyst was shaken at room temperature under 50 psig of H₂ for 8 hours. After removal of the catalyst by filtration, the solution was concentrated to dryness and the residue was recrystallized from acetonitrile to afford 0.119 g (75%) of Sa as a crystalline salt: mp 243-244° C.; CIMS (NH₃, M+1) 296; ¹H-NMR (CDCl₃, free base) δ 7.46 (d, 1, ArH, J=6.1 Hz), 7.24 (m, 3, ArH), 6.91 (s, 1, ArH), 6.74 (s, 1, ArH), 4.09 (s, 2, ArCH₂N), 3.88 (s, 3, OCH₃), 3.78 (m, 4, OCH₃, Ar₂CH), 2.87 (m, 3, CHN, ArCH₂), 2.17 (m, 1, CH₂CN), 1.61 (m, 2, NH, CH₂CN).

Trans-10,11-dihydroxy-5,6,6a,7,8,12b-hexahydrobenzo[α]phenanthridine hydrochloride (dihydrexidine, 6a). A suspension of 0.109 g (0.33 mmol) of the 10,11-dimethoxy salt 5a, in 1.5 mL of 48% HBr, was heated at reflux, under N₂, for 3 hours. The reaction mixture was concentrated to dryness under high vacuum. This material was dissolved in water and neutralized to the free base with NaHCO₃, while cooling the solution in an ice bath. The free base was extracted into chloroform, dried, filtered, and concentrated in vacuo. The residue was dissolved in ethanol and carefully neutralized with conc. HCl. After removal of the volatiles, the salt was crystallized as a solvate from methanol. This afforded 30 mg (25.2%) of 6, solvated with a stoichiometry of 1 molecule of amine salt and 1.8 molecules of CH₃OH, as pale yellow crystals: mp 195° C.; CIMS (isobutane, M+1) 268; ¹H-NMR (DMSO, HBr salt) δ 9.40 (bs, 1, ⁺NH₂), 9.22 (bs, 1, +NH₂), 8.76 (bs, 2, OH), 7.38 (m, 4, ArH), 6.72 (s, 1, ArH), 6.63 (s, 1, ArH), 4.40 (s, 2, ArCH₂N⁺), 4.16 (d, 1, Ar₂CH, J=11.1 Hz), 3.00 (m, 1, CHN⁺), 2.75 (m, 2, ArCH₂), 2.17 (m, 1, CH₂CN⁺), 1.90 (m, 1, CH₂CN⁺).

### Example 2

### 2-Methyldihydrexidine (6b)

2-(N-benzyl-N-4-methylbenzoyl)-6,7-dimethoxy-3,4-dihydro-2-naphthylamine (2b). To a solution of 4.015 g (19.5 mmol) of 6,7-dimethoxy-β-tetralone in 100 mL of toluene was added 2.139 g (1.025 equiv.) of benzylamine. The reaction was heated at reflux overnight under N₂ with continuous water removal. The reaction was cooled and the solvent was removed to yield N-benzyl enamine as a brown oil.

The 4-methylbenzoyl chloride acylating agent was prepared by suspending 3.314 g (24.3 mmol) of 4-toluic acid in 200 mL benzene. To this solution was added 2.0 equivalents (4.25 mL) of oxalyl chloride, dropwise via a pressure-equalizing dropping funnel at 0° C. Catalytic DMF (2-3 drops) was added to the reaction mixture and the ice bath was removed. The progress of the reaction was monitored using infrared spectroscopy. The solvent was removed and the residual oil was held under high vacuum overnight.

The resulting N-benzyl enamine residue was dissolved in 100 mL of CH₂Cl₂, and to this solution was added 2.02 g (19.96 mmol) of triethylamine at 0° C. The 4-methylbenzoyl chloride (3.087 g, 19.96 mmol) was dissolved in 20 mL CH₂Cl₂ and this solution was added dropwise to the cold, stirring N-benzyl enamine solution. The reaction was allowed to warm to room temperature and was left to stir under N₂ overnight. The reaction mixture was washed successively with 2×30 mL of 5% aqueous HCl, 2×30 mL of saturated sodium bicarbonate solution, saturated NaCl solution, and was dried over MgSO₄. After filtration, the filtrate was concentrated. Crystallization from diethyl ether gave 5.575 g (69.3%) of the enamide 2b: mp 96-98° C.; CIMS (isobutane, M+1) 414; ¹H-NMR (CDCl₃) δ 7.59 (d, 2, ArH), 7.46 (m, 3, ArH), 7.35 (m, 3, ArH), 7.20 (d, 2, ArH), 6.60 (s, 1, ArH), 6.45 (s, 1, ArH), 6.18 (s, 1, ArCH), 5.01 (s, 2, ArCH₂N), 3.80 (S, 3, OCH₃), 3.78 (s, 3, OCH₃), 2.53 (t, 2, ArCH₂), 2.37 (s, 3, ArCH₃), 2.16 (t, 2, CH₂).

Trans-2-methyl-6-benzyl-10,11-dimethoxy-5,6,6a,7,8,12b-hexahydrobenzo[α]phenanthridine-5-one (3b). A solution of 4.80 g (11.62 mmol) of the 6,7-dimethoxyenamide 2b, in 500 mL of THF, was introduced to an Ace Glass 500 mL photochemical reactor. This solution was stirred while irradiating for 2 hours with a 450 watt Hanovia medium pressure, quartz, mercury-vapor lamp seated in a water cooled, quartz immersion well. The solution was concentrated and crystallized from diethyl ether to provide 2.433 (50.7%) of the 10,11-dimethoxy lactam 3b: mp 183-195° C.; CIMS (isobutane, M+1) 414; ¹H-NMR (CDCl₃) δ 8.13 (d, 1, ArH), 7.30 (s, 1, ArH), 7.23 (m, 6, ArH), 6.93 (s, 1, ArH), 6.63 (s, 1, ArH), 5.38 (d, 1, ArCH₂N), 5.30 (d, 1, ArCH₂N), 4.34 (d, 1, Ar₂CH, J=11.4 Hz), 3.89 (s, 3, OCH₃), 3.88 (s, 3, OCH₃), 3.76 (m, 1, CHN), 2.68 (m, 2, ArCH₂), 2.37 (s, 3, ArCH₃), 2.25 (m, 1, CH₂CN), 1.75 (m, 1, CH₂CN).

Trans-2-methyl-6-benzyl-10,11-dimethoxy-5,6,6a,7,8,12b-hexahydrobenzo[α]phenanthridine hydrochloride (4b). A solution of 1.349 g (3.27 mmol) of the lactam 3b, in 100 mL dry THF was cooled in an ice-salt bath and 4.0 equivalents (13.0 mL) of 1.0 molar BH₃ was added through a syringe. The reaction was heated at reflux under nitrogen overnight. Methanol (10 mL) was added dropwise to the reaction mixture and reflux was continued for 1 hour. The solvent was removed. The residue was chased two times with methanol and twice with ethanol. The residue was placed under high vacuum (0.05 mm Hg) overnight. The residue was dissolved in ethanol and was carefully acidified with concentrated HCl. The volatiles were removed and the product was crystallized from ethanol to afford 1.123 g (78.9%) of the hydrochloride salt 4b: mp 220-223° C.; CIMS (isobutane, M+1) 400; ¹H-NMR (CDCl₃, free base) δ 7.37 (d, 2, ArH), 7.33 (m, 2, ArH), 7.26 (m, 1, ArH), 7.22 (s, 1, ArH), 7.02 (d, 1, ArH), 6.98 (d, 1, ArH), 6.89 (s, 1, ArH), 6.72 (s, 1, ArH), 4.02 (d, 1, Ar₂CH, J=10.81 Hz), 3.88 (s, 3, OCH₃), 3.86 (d, 1, ArCH₂N), 3.82 (m, 1, ArCH₂N), 3.78 (s, 3, OCH₃), 3.50 (d, 1, ArCH₂N), 3.30 (d, 1, ArCH₂N), 2.87 (m, 1, ArCH₂), 2.82 (m, 1, CHN), 2.34 (m, 1, CH₂CN), 2.32 (s, 3, ArCH₃), 2.20 (m, 1, ArCH₂), 1.93 (m, 1, CH₂CN).

Trans-2-methyl-10,11-dimethoxy-5,6,6a,7,8,12b-hexahydrobenzo[α]phenanthridine hydrochloride (5b). A solution of 0.760 g (1.75 mmol) of the 6-benzyl derivative 4b in 100 mL of 95% ethanol containing 150 mg of 10% Pd/C catalyst was shaken at room temperature under 50 psig of H₂ for 8 hours. After removal of the catalyst by filtration through Celite, the solution was concentrated to dryness and the residue was recrystallized from acetonitrile to afford 0.520 g (86.2%) of 5b as a crystalline salt: mp 238-239° C.; CIMS (isobutane, M+1) 310; ¹H-NMR (DMSO, HCl salt) δ 10.04 (s, 1, NH), 7.29 (d, 1, ArH), 7.16 (m, 2, ArH), 6.88 (s, 1, ArH), 6.84 (s, 1, ArH), 4.31 (s, 2, ArCH₂N), 4.23 (d, 1, Ar₂CH, J=10.8 Hz), 3.76 (s, 3, OCH₃), 3.70 (s, 3, OCH₃), 2.91 (m, 2, ArCH₂), 2.80 (m, 1, CHN), 2.49 (s, 3, ArCH₃), 2.30 (m, 1, CH₂CN), 2.09 (m, 1, CH₂CN).

Trans-2-methyl-10,11-dihydroxy-5,6,6a,7,8,12b-hexahydrobenzo [α]phenanthridine hydrochloride (6b). The 10,11-dimethoxy hydrochloride salt 5b (0.394 g, 1.140 mmol) was converted to its free base. The free base was dissolved in 35 mL of CH₂Cl₂ and the solution was cooled to −78° C. A 1.0 molar solution of BBr₃ (4.0 eq., 4.56 mL) was added slowly through a syringe. The reaction was stirred under N₂ overnight with concomitant warming to room temperature. Methanol (7.0 mL) was added to the reaction mixture and the solvent was removed. The residue was placed under high vacuum (0.05 mm Hg) overnight. The residue was dissolved in water and was carefully neutralized to its free base initially with sodium bicarbonate and finally with ammonium hydroxide (1-2 drops). The free base was isolated by suction filtration and was washed with cold water. The filtrate was extracted several times with dichloromethane and the organic extracts were dried, filtered, and concentrated. The filter cake and the organic residue were combined, dissolved in ethanol, and carefully acidified with concentrated HCl. After removal of the volatiles, the HCl salt was crystallized as a solvate from methanol in a yield of 0.185 g (51%) of 6b: mp 190° C. (dec.); CIMS (isobutane, M+1) 282; ¹H-NMR (DMSO, HCl salt) δ 9.52 (s, 1, NH), 8.87 (d, 2, OH), 7.27 (d, 1, ArH), 7.20 (s, 1, ArH), 7.15 (d, 1, ArH), 6.72 (s, 1, ArH), 6.60 (s, 1, ArH), 4.32 (s, 2, ArCH₂N), 4.10 (d, 1, ArCH₂CH, J=11.26 Hz), 2.90 (m, 1, CHN), 2.70 (m, 2, ArCH₂), 2.32 (s, 3, ArCH₃), 2.13 (m, 1, CH₂CN), 1.88 (m, 1, CH₂CN).

### Example 3.

### 3-Methyldihydrexidine (6c)

2-(N-benzyl-N-3-methylbenzoyl)-6,7-dimethoxy-3,4-dihydro-2-naphthylamine (2c). To a solution of 3.504 g (17.0 mmol) of 6,7-dimethoxy-β-tetralone in 100 mL of toluene was added 1.870 g (1.025 equivalents) of benzylamine. The reaction was heated at reflux overnight under N₂ with continuous water removal. The reaction was cooled and the solvent was removed to yield the N-benzyl enamine as a brown oil.

The 3-methylbenzoyl chloride acylating agent was prepared by suspending 3.016 g (22.0 mmol) of 3-toluic acid in 100 mL benzene. To this solution was added 2.0 equivalents (3.84 mL) of oxalyl chloride, dropwise with a pressure-equalizing dropping funnel at 0° C. Catalytic DMF (2-3 drops) was added to the reaction mixture and the ice bath was removed. The progress of the reaction was monitored using infrared spectroscopy. The solvent was removed and the residual oil was held under high vacuum overnight.

The resulting N-benzyl enamine residue was dissolved in 100 mL of CH₂Cl₂, and to this solution was added 1.763 g (17.42 mmol) of triethylamine at 0° C. The 3-methylbenzoyl chloride (2.759 g, 17.84 mmol) was dissolved in 20 mL CH₂Cl₂ and this solution was added dropwise to the cold, stirring N-benzyl enamine solution. The reaction was allowed to warm to room temperature and was left to stir under N₂ overnight. The reaction mixture was washed successively with 2×30 mL of 5% aqueous HCl, 2×30 mL of saturated sodium bicarbonate solution, saturated NaCl solution, and was dried over MgSO₄. After filtration, the filtrate was concentrated. Crystallization from diethyl ether gave 4.431 g (63.1%) of the enamide 2c: mp 96-97° C.; CIMS (isobutane, M+1) 414; ¹H-NMR (CDCl₃) δ 7.36 (s, 1, ArH), 7.26 (m, 3, ArH), 7.20 (m, 5, ArH), 6.50 (s, 1, ArH), 6.40 (s, 1, ArH), 6.05 (s, 1, ArCH), 4.95 (s, 2, ArCH₂N), 3.75 (s, 3, OCH₃), 3.74 (s, 3, OCH₃), 2.43 (t, 2, ArCH₂), 2.28 (s, 3, ArCH₃), 2.07 (t, 2, CH₂).

Trans-3-methyl-6-benzyl-10,11-dimethoxy-5,6,6a,7,8,12b-hexahydrobenzo[α]phenanthridine-5-one (3c). A solution of 1.922 g (4.65 mmol) of the 6,7-dimethoxyenamide 2c, in 500 mL of THF, was introduced to an Ace Glass 500 mL photochemical reactor. This solution was stirred while irradiating for 5 hours with a 450 watt Hanovia medium pressure, quartz, mercury-vapor lamp seated in a water-cooled, quartz immersion well. The solution was concentrated and crystallized from diethyl ether to provide 0.835 g (43.4%) of lactam 3c: mp 154-157° C.; CIMS (isobutane, M+1) 414; ¹H-NMR (CDCl₃) δ 7.94 (s, 1, ArH), 7.34 (d, 1, ArH), 7.17 (m, 6, ArH), 6.84 (s, 1, ArH), 6.54 (s, 1, ArH), 5.28 (d, 1, ArCH₂N), 4.66 (d, 1, ArCH₂N), 4.23 (d, 1, Ar₂CH, J=11.4 Hz), 3.78 (s, 3, OCH₃), 3.74 (s, 3, OCH₃), 3.61 (m, 1, CHN), 2.59 (m, 2, ArCH₂), 2.34 (s, 3, ArCH₃), 2.15 (m, 1, CH₂CN), 1.63 (m, 1, CH₂CN).

Trans-3-methyl-6-benzyl-10,11-dimethoxy-5,6,6a,7,8,12b-hexahydrobenzo[α]phenanthridine hydrochloride (4c). A solution of 0.773 g (1.872 mmol) of the lactam 3c, in 50 mL dry THF was cooled in an ice-salt bath and 4.0 equivalents (7.5 mL) of 1.0 molar BH₃ were added through a syringe. The reaction was heated at reflux under N₂ overnight. Methanol (6 mL) was added dropwise to the reaction mixture and reflux was continued for 1 hr. The solvent was removed. The residue was chased two times with methanol and twice with ethanol. The residue was placed under high vacuum (0.05 mm Hg) overnight. The residue was dissolved in ethanol and was carefully acidified with concentrated HCl. The volatiles were removed and the product was crystallized from ethanol to afford 0.652 g (80%) of 4c as the hydrochloride salt: mp 193-195° C.; CIMS (isobutane, M+1) 400; ¹H-NMR (CDCl₃, free base) δ 7.38 (d, 2, ArH), 7.33 (m, 2, ArH), 7.28 (m, 2, ArH), 7.07 (d, 1, ArH), 6.90 (s, 1, ArH), 6.88 (s, 1, ArH), 6.72 (s, 1, ArH), 4.02 (d, 1, Ar₂CH, J=11.2 Hz), 3.90 (d, 1, ArCH₂N), 3.87 (s, 3, OCH₃), 3.82 (m, 1, ArCH₂N), 3.78 (s, 3, OCH₃), 3.48 (d, 1, ArCH₂N), 3.30 (d, 1, ArCH₂N), 2.88 (m, 1, ArCH₂), 2.82 (m, 1, CHN), 2.36 (m, 1, CH₂CN), 2.32 (s, 3, ArCH₃), 2.20 (m, 1, ArCH₂), 1.95 (m, 1, CH₂CN).

Trans-3-methyl-10, 11-dimethoxy-5,6,6a,7,8,12b-hexahydrobenzo[α]phenanthridine hydrochloride (5c). A solution of 0.643 g (1.47 mmol) of the 6-benzyl hydrochloride salt 4c prepared above in 100 mL of 95% ethanol containing 130 mg of 10% Pd/C catalyst was shaken at room temperature under 50 psig of H₂ for 8 hours. After removal of the catalyst by filtration through Celite, the solution was concentrated to dryness and the residue was recrystallized from acetonitrile to afford 0.397 g (78%) of 5c as a crystalline salt: mp 254-256° C.; CIMS (isobutane, M+1) 310; ¹H-NMR (DMSO, HCl salt) δ 10.01 (s, 1, NH), 7.36 (d, 1, ArH), 7.09 (d, 1, ArH), 6.98 (s, 1, ArH), 6.92 (s, 1, ArH), 6.74 (s, 1, ArH), 4.04 (s, 2, ArCH₂N), 3.88 (s, 3, OCH₃), 3.81 (s, 3, OCH₃), 3.76 (d, 1, Ar₂CH), 2.89 (m, 2, ArCH₂), 2.70 (m, 1, CHN), 2.36 (s, 3, ArCH₃), 2.16 (m, 1, CH₂CN), 1.70 (m, 1, CH₂CN).

Trans-3-methyl-10,11-dihydroxy-5,6,6a,7,8, 12b-hexahydrobenzo[α]phenanthridine hydrochloride (6c). The 10,11-dimethoxy hydrochloride salt 5c (0.520 g, 1.51 mmol) was converted to its free base. The free base was dissolved in 35 mL of dichloromethane and the solution was cooled to −78° C. A 1.0 molar solution of BBr₃ (4.0 equivalents, 6.52 mL) was added slowly via syringe. The reaction was stirred under N₂ overnight with concomitant warming to room temperature. Methanol (7.0 mL) was added to the reaction mixture and the solvent was removed. The residue was placed under high vacuum (0.05 mm Hg) overnight. The residue was dissolved in water and was carefully neutralized to its free base initially with sodium bicarbonate and finally with ammonium hydroxide (1-2 drops). The free base was isolated by suction filtration and was washed with cold water. T he filtrate was extracted several times with dichloromethane and the organic extracts were dried, filtered, and concentrated. The filter cake and the organic residue were combined, dissolved in ethanol, and carefully acidified with concentrated HCl. After removal of the volatiles, the HCl salt was crystallized as a solvate from methanol to yield 0.341 g (71.3%) or 6c: mp 190° C. (dec.); CIMS (isobutane, M+1) 282; ¹H-NMR (DMSO, HCl salt) δ 9.55 (s, 1, NH), 8.85 (d, 2, OH), 7.30 (d, 1, ArH), 7.22 (s, 1, ArH), 7.20 (d, 1, ArH), 6.68 (s, 1, ArH), 6.60 (s, 1, ArH), 4.31 (s, 2, ArCH₂N), 4.09 (d, 1, ArCH₂CH, J=11.2 Hz), 2.91 (m, 1, CHN), 2.72 (m, 2, ArCH₂), 2.35 (s, 3, ArCH₃), 2.16 (m, 1, CH₂CN, 1.85 (m, 1, CH₂CN).

### Example 4

### 4-Methyldihydrexidine (6d)

2-(N-benzyl-N-2-methylbenzoyl)-6,7-dimethoxy-3,4-dihydro-2-naphthylamine (2d). To a solution of 5.123 g (24.8 mmol) of 6,7-dimethoxy-β-tetralone in 200 mL of toluene was added 2.929 g (1.025 equivalents) of benzylamine. The reaction was heated at reflux overnight under N₂ with continuous water removal. The reaction was cooled and the solvent was removed to yield the N-benzyl enamine as a brown oil.

The 2-methylbenzoyl chloride acylating agent was prepared by suspending 4.750 g (42.2 mmol) of 2-toluic acid in 100 mL benzene. T o this solution was added 2.0 equivalents (7.37 mL) of oxalyl chloride, dropwise via a pressure-equalizing dropping funnel at 0° C. Catalytic DMF (2-3 drops) was added to the reaction mixture and the ice bath was removed. The progress of the reaction was monitored using infrared spectroscopy. The solvent was removed and the residual oil was held under high vacuum overnight.

The resulting N-benzyl enamine residue was dissolved in 100 mL of CH₂Cl₂, and to this solution was added 2.765 g (1.1 equivalent) of triethylamine at 0 C. The 2-methylbenzoyl chloride (4.226 g, 27.3 mmol) was dissolved in 25 mL CH₂Cl₂ and this solution was added dropwise to the cold, stirring N-benzyl enamine solution. The reaction was allowed to warm to room temperature and was left to stir under N₂ overnight. The reaction mixture was washed successively with 2×30 mL of 5% aqueous HCl, 2×30 mL of saturated sodium bicarbonate solution, saturated NaCl solution, and was dried over MgSO₄. After filtration, the filtrate was concentrated. The resulting oil was purified via a chromatotron utilizing a 5% ether/dichloromethane eluent mobile phase to yield 3.950 g (38.5%) of 2d as an oil: CIMS (isobutane, M+1) 414; 1H-NMR (CDCl₃) δ 7.34 (d, 2, ArH), 7.30 (m, 2, ArH), 7.25 (d, 2, ArH), 7.14 (m, 2, ArH), 7.07 (m, 1, ArH), 6.47 (s, 1, ArH), 6.37 (s, 1, ArH), 6.04 (s, 1, ArCH), 4.96 (s, 2, ArCH₂N), 3.78 (s, 3, OCH₃), 3.77 (s, 3, OCH₃), 2.39 (s, 3, ArCH₃), 2.30 (t, 2, ArCH₂), 1.94 (t, 2, CH₂).

Trans-4-methyl-6-benzyl-10,11-dimethoxy-5,6,6a,7,8, 12b-hexahydrobenzo[α]phenanthridine-5-one (3d). A solution of 2.641 g (6.395 mmol) of the 6,7-dimethoxyenamide 2d, in 450 mL of THF, was introduced to an Ace Glass 500 mL photochemical reactor. This solution was stirred while irradiating for 3 hours with a 450 watt Hanovia medium pressure, quartz, mercury-vapor lamp seated in a water-cooled, quartz immersion well. The solution was concentrated and crystallized from diethyl ether to provide 0.368 (20%) of the 10,11-dimethoxy lactam 3d: mp 175-176° C.; CIMS (isobutane, M+1) 414; 1H-NMR (CDCl₃) δ 7.88 (m, 3, ArH), 7.65 (d, 1, ArH), 7.40 (m, 2, ArH), 7.21 (m, 2, ArH), 6.87 (s, 1, ArH), 6.60 (s, 1, ArH), 5.34 (d, 1, ArCH₂N), 4.72 (d, 1, ArCH₂N), 4.24 (d, 1, Ar₂CH, J=10.9 Hz), 3.86 (s, 3, OCH₃), 3.85 (s, 3, OCH₃), 3.68 (m, 1, CHN), 2.73 (s, 3, ArCH₃), 2.64 (m, 2, ArCH₂); 2.20 (m, 1, CH₂CN), 1.72 (m, 1, CH₂CN).

Trans-4-methyl-6-benzyl-10, 11-dimethoxy-5,6,6a,7,8, 12b-hexahydrobenzo[α]phenanthridine hydrochloride (4d). A solution of 1.640 g (3.97 mmol) of the lactam 3d, in 100 mL dry THF was cooled in an ice-salt bath and 4.0 equivalents (15.9 mL) of 1.0 molar BH₃ were added through a syringe. The reaction was heated at reflux under N₂ overnight. Methanol (10 mL) was added dropwise to the reaction mixture and reflux was continued for 1 hour. The solvent was removed and the residue was chased two times with methanol and twice with ethanol. The residue was placed under high vacuum (0.05 mm Hg) overnight. The residue was dissolved in ethanol and was carefully acidified with concentrated HCl. The volatiles were removed and the product was crystallized from ethanol to afford 1.288 g (74.5%) of 4d as the hydrochloride salt: mp 232-235° C.; CIMS (isobutane, M+1), 400; ¹H-NMR (CDCl₃, free base) δ 7.38 (d, 2, ArH), 7.33 (m, 2, ArH), 7.27 (d, 1, ArH), 7.24 (m, 1, ArH), 7.16 (m, 1, ArH), 7.06 (d, 1, ArH), 6.85 (s, 1, ArH), 6.71 (s, 1, ArH), 4.05 (d, 1, Ar₂CH, J=10.8 Hz), 3.89 (d, 1, ArCH₂N), 3.87 (s, 3, OCH₃), 3.82 (m, 1, ArCH₂N), 3.76 (s, 3, OCH₃), 3.55 (d, 1, ArCH₂N), 3.31 (d, 1, ArCH₂N), 2.88 (m, 1, ArCH₂), 2.81 (m, 1, CHN), 2.34 (m, 1, CH₂CN), 2.20 (m, 1, ArCH₂), 2.17 (s, 3, ArCH₃), 1.94 (m, 1, CH₂CN).

Trans-4-methyl-10, 11-dimethoxy-5,6,6a,7,8,12b-hexahydrobenzo[α]phenanthridine hydrochloride (5d). A solution of 0.401 g (0.92 mmol) of the 6-benzyl hydrochloride salt 4d in 100 mL of 95% ethanol containing 100 mg of 10% Pd/C catalyst was shaken at room temperature under 50 psig of H₂ for 8 hours. After removal of the catalyst by filtration through Celite, the solution was concentrated to dryness and the residue was recrystallized from acetonitrile to afford 0.287 g (90.2%) of Sd as a crystalline salt: mp 215-216° C.; CIMS (isobutane, M+1) 310; ¹H-NMR (CDCl₃, free base) δ 9.75 (s, 1, NH), 7.29 (d, 1, ArH), 7.28 (d, 1, ArH), 7.21 (m, 1, ArH), 6.86 (s, 1, ArH), 6.81 (s, 1, ArH), 4.35 (d, 1, ArCH₂N), 4.26 (d, 1, ArCH₂N), 4.23 (d, 1, Ar₂CH, J=11.17 Hz), 3.75 (s, 3, OCH₃), 3.65 (s, 3, OCH₃), 2.96 (m, 1, CHN), 2.83 (m, 2, ArCH₂), 2.30 (s, 3, ArCH₃), 2.21 (m, 1, CH₂CN), 1.93 (m, 1, CH₂CN).

Trans-4-methyl-10,11-dihydroxy-5,6,6a,7,8,12b-hexahydrobenzo[α]phenanthridine hydrochloride (6d). The 10,11-dimethoxy hydrochloride salt 5d (0.485 g, 1.40 mmol) was converted to its free base. The free base was dissolved in 35 mL of dichloromethane and the solution was cooled to −78° C. A 1.0 molar solution of BBr₃ (4.0 equivalents, 5.52 mL) was added slowly through a syringe. The reaction was stirred under N₂ overnight with concomitant warming to room temperature. Methanol (7.0 mL) was added to the reaction mixture and the solvent was removed. The residue was placed under high vacuum (0.05 mm Hg) overnight. The residue was dissolved in water and was carefully neutralized to its free base initially with sodium bicarbonate and finally with ammonium hydroxide (1-2 drops). The free base was isolated by suction filtration and was washed with cold water, the filtrate was extracted several times with dichloromethane and the organic extracts were dried, filtered, and concentrated. The filter cake and the organic residue were combined, dissolved in ethanol and carefully acidified with concentrated HCl. After removal of the volatiles, the HCl salt was crystallized as a solvate from methanol to yield 0.364 g (81.6%) of 6d: mp 195° C. (dec.); CIMS (isobutane, M+1) 282; ¹H-NMR (DMSO, HCl salt) d 9.55 (s, 1, NH), 8.85 (s, 1, OH), 8.80 (s, 1, OH), 7.28 (m, 2, ArH), 7.20 (d, 1, ArH), 6.65 (s, 1, ArH), 6.60 (s, 1, ArH), 4.32 (d, 1, ArCH₂N), 4.26 (d, 1, ArCH₂N), 4.13 (d, 1, Ar₂CH, J=11.63 Hz), 2.92 (m, 1, CHN), 2.75 (m, 1, ArCH₂), 2.68 (m, 1, ArCH₂), 2.29 (s, 3, ArCH₃), 2.17 (m, 1, CH₂CN), 1.87 (m, 1, CH₂CN).

### Example 5

### 2-Benzyldihydrexidine (6e)

Trans-2-benzyl-10,11-dihydroxy-5,6,6a,7,8,12b-hexahydrobenzo[α]phenanthridine hydrochloride (6e) prepared according to the procedure described in Example 4, except that 4-methylbenzoyl chloride was replaced with 2-benzylbenzoyl chloride.

### Example 6

### Dinoxyline (16a).

1,2-Dimethoxy-3-methoxymethoxybenzene (8). A slurry of sodium hydride was prepared by adding 1000 mL of dry THF to 7.06 g (0.18 mol) of sodium hydride (60% dispersion in mineral oil) under an argon atmosphere at 0° C. To the slurry, 2,3-dimethoxyphenol (7) (23.64 g, 0.153 mol) was added through a syringe. The resulting solution was allowed to warm to room temperature and stirred for two hours. The resulting black solution was cooled to 0° C. and 13.2 mL of chloromethylmethyl ether (14 g, 0.173 mol) was slowly added with a syringe. The solution was allowed to reach room temperature and stirred for an additional 8 hours. The resulting yellow mixture was concentrated to an oil that was dissolved in 1000 mL of diethyl ether. The resulting solution was washed with water (500 mL), 2N NaOH (3×400 mL), dried (MgSO₄), filtered, and concentrated. After Kugelrohr distillation (90-100° C., 0.3 atm), 24.6 g (84%) of 8 as a clear oil was obtained: ¹H NMR (300 MHz, CDCl₃) δ 6.97 (t, 1H, J=8.7 Hz); 6.79 (dd, 1H, J=7.2, 1.8 Hz); 6.62 (dd, 1H, J=6.9, 1.2 Hz); 5.21 (s, 2H); 3.87 (s, 3H); 3.85 (s, 3H); 3.51 (s, 3H); CIMS m/z 199 (M+H⁺, 50%); 167 (M+H⁺, CH₃0H, 100%); Anal. Calc'd for C₁₀H₁₄O₄: C, 60.59; H, 7.12. Found: C, 60.93; H, 7.16.

2-(3,4-Dimethoxy-2-methoxymethoxyphenyl)-4,4,5,5-tetra-methyl-1,3,2-dioxaborolane (9). The MOM-protected phenol 8 (10 g, 0.0505 mol) was dissolved in 1000 mL of dry diethyl ether and cooled to −78° C. A solution of n-butyl lithium (22.2 mL, 2.5 M) was then added with a syringe. The cooling bath was removed and the solution was allowed to warm to room temperature. After stirring the solution at room temperature for two hours, a yellow precipitate was observed. The mixture was cooled to −78° C., and 15 mL of 2-isopropoxy-4,4,5,5-tetramethyl-1,3,2-dioxaborolane (0.080 mol) was added through a syringe. The cooling bath was removed after two hours. Stirring was continued for four hours at room temperature. The mixture was then poured into 300 mL of water and extracted several times with diethyl ether (3×300 mL), dried (Na₂SO₄), and concentrated to 9 a yellow oil (12.37g, 76%) that was used without further purification: ¹H NMR (300 MHz, CDCl₃) 6 7.46 (d, 1H, J=8.4 Hz); 6.69 (d, 1H, J=8.4 Hz); 5.15 (s, 2H); 3.87 (s, 3H); 3.83 (s, 3 H); 1.327 (s, 12H).

4-Bromo-5-nitroisoquinoline (11). Potassium nitrate (5.34 g; 0.052 mol) was added to 20 mL of concentrated sulfuric acid and slowly dissolved by careful heating. The resulting solution was added dropwise to a solution of 4-bromoisoquinoline (10 g, 0.048 mol) dissolved in 40 mL of the same acid at 0° C. After removal of the cooling bath, the solution was stirred for one hour at room temperature. The reaction mixture was then poured onto crushed ice (400 g) and made basic with ammonium hydroxide. The resulting yellow precipitate was collected by filtration and the filtrate was extracted with diethyl ether (3×500 mL), dried (Na₂SO₄), and concentrated to give a yellow solid that was combined with the initial precipitate. Recrystallization from methanol gave 12.1 g (89%) of 11 as slightly yellow crystals: mp 172-174° C.; ¹H NMR (300 MHz, CDCl₃) δ 9.27 (s, 1H); 8.87 (s, 1H); 8.21 (dd, 1H, J=6.6, 1.2 Hz); 7.96 (dd, 1 H, J=6.6, 1.2 Hz); 7.73 (t, 1 H, J=7.5 Hz); CIMS ml/z 253 (M+H⁺, 100%); 255 (M+H++2, 100%); Anal. Calc'd for C₉H₅BrN₂O₂: C, 42.72; H, 1.99; N, 11.07. Found: C, 42.59; H, 1.76; N, 10.87.

4-(3,4-Dimethoxy-2-methoxymethoxyphenyl)-5-nitroisoquinoline (12). Isoquinoline 11 (3.36 g, 0.0143 mol), pinacol boronate ester 9 (5.562 g, 0.0172 mol), and 1.0 g (6 mol %) of (Ph₃)Pd were suspended in 100 mL of dimethoxyethane (DME). Potassium hydroxide (3.6 g; 0.064 mol), and 0.46 g (10 mol %) of tetrabutylammonium bromide were dissolved in 14.5 mL of water and added to the DME mixture. The resulting suspension was degassed for 30 minutes with argon and then heated at reflux for four hours. The resulting black solution was allowed to cool to room temperature, poured into 500 mL of water, extracted with diethyl ether (3×500 mL), dried (Na₂SO₄), and concentrated. The product was then purified by column chromatography (silica gel, 50% ethyl acetate-hexane) giving 5.29 g (80.1%) of 12 as yellow crystals: mp 138-140° C.; ¹H NMR (300 MHz, CDCl₃) δ 9.33 (s, 1H); 8.61 (s, 1H); 8.24 (dd, 1H, J=7.2, 0.9 Hz); 8.0 (dd, 1H, J=6.3, 1.2 Hz); 7.67 (t, 1H, J =7.8 Hz); 7.03 (d, 1H, J=9.6 Hz); 6.81 (d, 1H, J=8.1 Hz); 4.86 (d, 1H, J=6 Hz); 4.70 (d, 1H, J=5.4 Hz); 3.92 (s, 3H); 3.89 (s, 3 H); 2.613 (s, 3 H); CIMS m/z 371 (M+H⁺, 100%); Anal. Calc'd for C₁₉H₁₈N₂O₆: C, 61.62; H, 4.90; N, 7.56. Found: C, 61.66; H, 4.90; N, 7.56.

2,3-Dimethoxy-6-(5-nitroisoquinolin-4-yl)phenol (13). After dissolving isoquinoline 12 (5.285 g, 0.014 mol) in 200 mL of methanol by gentle heating, p-toluenesulfonic acid monohydrate (8.15 g; 0.043 mol) was added in several portions. Stirring was continued for four hours at room temperature. After completion of the reaction, the solution was made basic by adding saturated sodium bicarbonate. The product was then extracted with CH₂Cl₂ (3×250 mL), dried (Na₂SO₄), and concentrated. The resulting 13 as a yellow solid (4.65 g; 98%) was used directly in the next reaction. An analytical sample was recrystallized from methanol: mp 170-174° C.; ¹H NMR (300 MHz, CDCl₃) δ 9.33 (s, 1H); 8.62 (s, 1H); 8.24 (dd, 1H, J=7.2, 0.9 Hz); 7.99 (dd, 1H, J=6.3, 1.2 Hz); 7.67 (t, 1H, J=7.8 Hz); 6.96 (d, 1H, J=8.7 Hz); 6.59 (d, 1H, J=8.7 Hz); 5.88 (bs, 1H); 3.94 (s, 3H); 3.92 (s, 3H); CIMS m/z 327 (M+H⁺, 100%); Anal. Calc'd for C₁₇H₁₄N₂O₅: C, 62.57; H, 4.32; N, 8.58; Found: C, 62.18; H, 4.38; N, 8.35.

8,9-dimethoxychromeno[4,3,2-de]isoquinoline (14). Phenol 13 (4.65 g, 0.014 mol) was dissolved in 100 mL of dry DMF. The solution was degassed with argon for thirty minutes. Potassium carbonate (5.80 g, 0.042 mol) was added to the yellow solution in one portion. After heating at 80° C. for one hour, the mixture had turned brown and no more starting material remained. After the solution was cooled to room temperature, 200 mL of water was added. The aqueous layer was extracted with dichloromethane (3×500 mL), this organic extract was washed with water (3 x 500 mL), dried (Na₂SO₄), and concentrated. Isoquinoline 14 was obtained as a white powder (3.65 g 92%) and was used in the next reaction without further purification. An analytical sample was recrystallized from ethyl acetate:hexane: mp 195-196° C.; ¹H NMR (300 MHz, CDCl₃) δ 9.02 (s, 1H); 8.82 (s, 1H); 7.87 (d, 1H, J=8.7 Hz); 7.62 (m, 3H); 7.32 (dd, 1H, J=6.0, 1.5 Hz); 6.95 (d, J=9.6 Hz); 3.88 (s, 3H); 3.82 (s, 3H). CIMS m/z 280 (M+H⁺, 100%). 8,9-dimethoxy-1,2,3,11b-tetrahydrochromeno[4,3,2-de]isoquinoline (15a). Platinum (IV) oxide (200 mg) was added to a solution containing 50 mL of acetic acid and isoquinoline 14 (1 g; 3.5 mmol). After adding 2.8 mL of concentrated HCl, the mixture was shaken on a Parr hydrogenator at 60 psi for 24 hours. The resulting green solution was filtered through Celite to remove the catalyst and the majority of the acetic acid was removed under reduced pressure. The remaining acid was neutralized using a saturated sodium bicarbonate solution, extracted with diethyl ether (3×250 mL), dried (Na₂SO₄), and concentrated. The resulting 14 as an oil (0.997 g; 99%) was used without further purification: ¹H NMR (300 MHz, CDCl₃) δ 7.10 (t, 1H, J=7.5 Hz); 7.00 (d, 1H, J=8.4 Hz); 6.78 (m, 2H); 6.60 (d, 1H, J=9 Hz); 4.10 (s, 2H); 3.84 (m, 8H); 2.93 (t, 1H, J 12.9 Hz).

8,9-dihydroxy-1,2,3,11b-tetrahydrochromeno[4,3,2-de]isoquinoline hydrobromide (16a). The dimethoxy derivative 15a (0.834 g; 3.0 mmol) was dissolved in 50 mL of anhydrous dichloromethane. The solution was cooled to −78° C. and 15.0 mL of a boron tribromide solution (1.0 M in dichloromethane) was slowly added. The solution was stirred overnight, while the reaction slowly warmed to room temperature. The solution was recooled to −78° C., and 50 mL of methanol was slowly added to quench the reaction. The solution was then concentrated to dryness. Methanol was added and the solution was concentrated. This process was repeated three times. The resulting brown solid was treated with activated charcoal and recrystallized from ethanol to give 16a: mp 298-302° C. (dec.); ¹H NMR (300 MHz, D₂O) δ 7.32 (t, 1H, J=6.6 Hz); 7.13 (d, 1H, J=8.4 Hz); 7.04 (d, 1H, J=8.4 Hz); 4.37 (m, 2H); 4.20 (t, 3H, J=10 Hz); Anal. Calc'd for C₁₅H₁₄BrNO₃.H₂O: C, 50.87; H, 4.55; N, 3.82. Found: C, 51.18; H, 4.31; N, 3.95.

### Example 7

### N-Allyl dinoxyline (16b)

N-allyl-8,9-dimethoxy-1,2,3,11b-tetrahydrochromeno[4,3,2-de]isoquinoline (15b). Tetrahydroisoquinoline 15a (1.273 g; 4.5 mmol) was dissolved in 150 mL of acetone. Potassium carbonate (0.613 g; 4.5 mmol) and 0.4 mL (4.6 mmol) of allyl bromide were added. The reaction was stirred at room temperature for four hours. The solid was then removed by filtration and washed on the filter several times with ether. The filtrate was concentrated and purified by flash chromatography (silica gel, 50% ethyl acetate-hexane) to give 1.033 g (71%) of 15b a yellow oil that was used without further purification: ¹H NMR (300 MHz, CDCl₃) δ 7.15 (t, 1H, J=9 Hz); 7.04 (d, 1H, J=9 Hz); 6.83 (m, 2H); 6.65 (d, 1H, J=6 Hz); 5.98 (m, 1H); 5.27 (m, 2H); 4.10 (m, 3H); 3.95 (s, 3H); 3.86 (s, 3H); 3.46 (d, 1H, J=15 Hz); 3.30 (d, 2H,J=6 Hz);2.56(t, 1H,J=12 Hz).

N-allyl-8,9-dihydroxy-1,2,3,11b-tetrahydrochromeno[4,3,2-de]isoquinoline (16b). N-Allylamine 15b (0.625 g; 1.93 mmol) was dissolved in 50 mL of dichloromethane. The solution was cooled to −78° C. and 10.0 mL of BBr₃ solution (1.0 M in dichloromethane) was slowly added. The solution was stirred overnight, while the reaction slowly warmed to room temperature. After recooling the solution to −78° C., 50 mL of methanol was slowly added to quench the reaction. The reaction was then concentrated to dryness. Methanol was added and the solution was concentrated. This process was repeated three times. Recystallization of the resulting brown solid from ethanol gave 0.68 g (61%) of 16b as a white solid: mp 251-253° C. (dec.); 1HNMR (300 MHz, D₂0) 8 10.55 (s, 1H); 10.16 (s, 1H); 8.61 (t, 1H, J=9 Hz); 8.42 (d, 1H, J=9 Hz); 8.31 (d, 1H, J=9 Hz); 7.87 (d, 1H, J=9 Hz); 7.82 (d, 1H, J=9 Hz); 7.36 (q, 1H, J=9 Hz); 6.89 (m, 2H); 6.85 (d, 1H, J=15 Hz); 5.58 (m, 3H); 5.28 (m, 2H); 3.76 (d, 1H, J=3 Hz). HRCIMS ml/z Calc'd: 295.1208; Found: 295.1214.

### Example 8

### N-Propyl dinoxyline (16c)

N-propyl-8,9-dimethoxy-1,2,3,11b-tetrahydrochromeno [4,3,2-de]isoquinoline (15c). N-Allylamine 15b (1.033 g; 3.2 mmol) was dissolved in 50 mL of ethanol. Palladium on charcoal (10% dry; 0.103 g) was then added. The mixture was shaken on a Parr hydrogenator under 60 psi H₂ for 3 hours. After TLC showed no more starting material, the mixture was filtered through Celite and concentrated to give 0.95 g (91%) of 15c as an oil that was used without further purification: ¹H NMR (300 MHz, CDCl₃) δ 7.15 (t, 1H, J=7.2 Hz); 7.04 (d, 1H, J=8.1 Hz); 6.84 (t, 2H, J=7.5 Hz); 6.65 (d, 1H, J=8.4 Hz); 4.07 (m, 2H); 3.95 (s, 3H); 3.86 (s, 3H); 3.71 (q, 1H, J=5.1 Hz); 3.42 (d, 2H, J=15.6 Hz); 2.62 (m, 2H); 2.471 (t, J=10.5 Hz); 1.69 (h, 2H, J=7.2 Hz); 0.98 (t, 3H, J=7.5 Hz); CIMS ml/z 326 (M+H+, 100%).

N-propyl-8,9-dihydroxy-1,2,3,11b-tetrahydrochromeno[4,3,2-de]isoquinoline (16c). The N-propyl amine 15c (0.90 g; 2.8 mmol) was dissolved in 200 mL of dichloromethane and cooled to −78° C. In a separate 250 mL round bottom flask, 125 mL of dry dichloromethane was cooled to −78° C., and 1.4 mL (14.8 mmol) of BBr₃ was added through a syringe. The BBr₃ solution was transferred using a cannula to the flask containing the starting material. The solution was stirred overnight, while the reaction slowly warmed to room temperature. After recooling the solution to −78° C., 50 mL of methanol was slowly added to quench the reaction. The reaction was then concentrated to dryness. Methanol was added and the solution was concentrated. This process was repeated three times. The resulting tan solid was suspended in hot isopropyl alcohol. Slowly cooling to room temperature resulted in a fine yellow precipitate. The solid was collected by filtration to give 16c (0.660 g; 63%): mp 259-264° C. (dec.); ¹H NMR (300 MHz, CDCl₃) δ 7.16 (t, 1H, J=9 Hz); 6.97 (d, 1H, J=12 Hz); 6.83 (d, 1H, J=9 Hz); 6.55 (d, 1H, J=9 Hz); 6.46 (d, 1H, J=9 Hz); 4.45 (d, 1H, J=15 Hz); 4.10 (m, 3H); 3.17 (q, 2H, J=6 Hz); 3.04 (t, 1H, J=9 Hz); 1.73 (q, 2H, J=9 Hz); 0.90 (t, 3H, J=6 Hz); Anal. Calc'd. for C₁₈H₂₀BrNO₃: C, 57.16; H, 5.33; N, 3.70. Found: C, 56.78; H, 5.26; N, 3.65.

### Example 9

### Preparation of 2-methyl-2,3-dihydro-4(1H)-isoquinolone (20)

Ethyl 2-bromomethylbenzoate (18). A solution of ethyl 2-toluate (17, 41.2 g, 0.25 mole) in carbon tetrachloride (200 mL) was added dropwise to a stirring mixture of benzoyl peroxide (100 mg), carbon tetrachloride (200 mL), and NBS (44.5 g, 0.25 mole) at 0° C. The mixture was heated at reflux for 3.5 hr under nitrogen, and allowed to cool to room temperature overnight. The precipitated succinimide was removed by filtration and the filter cake was washed with carbon tetrachloride. The combined filtrates were washed successively with 2 N NaOH (100 mL), and water (2×100 mL), and the solution was dried over anhydrous MgSO₄, filtered (Celite), and evaporated under vacuum to yield an oil. Drying under high vacuum overnight afforded 60.5 g (99%) of compound 18: ¹H NMR of the product showed the presence of ca. 15% of unreacted 17. The mixture was used in the next step without further purification: ¹H NMR (CDCl₃) δ 1.43 (t, J=7 Hz, 3H, CH₂ CH₃), 4.41 (q, J=7 Hz, 2H, CH₂CH₃), 4.96 (s, 1H, CH₂Br), 7.24 (m, 1H, ArH), 7.38 (m, 1H, ArH), 7.48 (m, 2H, ArH).

N-(2-carboethoxy)sarcosine ethyl ester (19). To a mixture of sarcosine ethyl ester hydrochloride (32.2 g, 0.21 mole), potassium carbonate (325 mesh; 86.9 g, 0.63 mole), and acetone (800 mL) was added a solution of compound 18 (60.7 g, ca. 0.21 mole, 85:15 18/17) in acetone (100 mL) at room temperature under N₂. The mixture was stirred at reflux for 2 hr and then left at room temperature for 20 hr. The solid was removed by filtration (Celite) and the residue was washed with acetone. The filtrates were combined and evaporated to afford an oil. The oil was dissolved in 250 mL of 3 N HCl and washed with ether. The aqueous layer was basified with aqueous NaHCO₃, and extracted with ether (3×250 mL). Evaporation of the ether solution yielded an oil that was vacuum distilled to afford 45.33 g (77%) of compound 19: bp 140-142° C./0.5 mm Hg; bp 182-183° C./10 mm Hg; ¹H NMR (CDCl₃) δ 1.24 (t, 3H, J=7.1 Hz, CH₃), 1.36 (t, 3H, J=7.1 Hz, CH₃), 2.35 (s, 3H, NCH₃), 3.27 (s, 2H, CH₂Ar), 4.00 (s, 2H, NCH₂), 4.14 (q, 2H, J=7.1 Hz, CH₂CH₃), 4.32 (q, 2H, J=7.1 Hz, CH₂CH₃), 7.28 (t, 1H, J=7.4 Hz, ArH), 7.42 (t, 1H, J=7.6 Hz, ArH), 7.52 (d, 1H, J=7.8 Hz, ArH), 7.74 (d, 1H, J=7.7 Hz, ArH).

2-Methyl-2,3-dihydro-4(1H)isoquinolone (20). Freshly cut sodium (10.9 g, 0.47 g-atom) was added to absolute ethanol (110 mL) under nitrogen and the reaction was heated at reflux. After the metallic sodium had disappeared, a solution of compound 19 (35.9 g, 0.128 mole) in dry toluene (160 mL) was added slowly to the reaction mixture. It was then heated at reflux and ethanol was separated azeotropically via a Dean Stark trap. After cooling, the solvent was evaporated under reduced pressure. The remaining yellow semi-solid residue was dissolved in a mixture of water (50 mL), 95% ethanol (60 mL), and concentrated HCl (240 mL), and heated at reflux for 26 hr. After cooling, the mixture was concentrated under vacuum and carefully basified with solid NaHCO₃. The basic solution was extracted with ether, dried (MgSO₄), and evaporated to an oil that was distilled to afford compound 20 (17.11 g, 83%): bp 130-132° C./5 mm Hg; bp 81-83° C./0.4 mm Hg; mp (HCl salt) 250° C.; IR (neat) 1694 (C═O) cm⁻¹; ¹H NMR (CDCl₃) δ 2.48 (s, 3H, CH₃), 3.31 (s, 2H, CH₂), 3.74 (s, 2 H, CH₂), 7.22 (d, 1H, J=7.7 Hz, ArH), 7.34 (t, 1H, J=7.9 Hz, ArH), 7.50 (t, 1H, J=7.5 Hz, ArH), 8.02 (d, 1H, J=7.9 Hz, ArH).

### Example 10

### Dinapsoline (29)

2′,3′-Dihydro-4,5-dimethoxy-2′-methylspiro[isobenzofuran-1(3H),4′(1′H)-isoquinoline]-3-one (22). To a solution of 2,3-dimethoxy-N′-diethylbenzamide (21, 14.94 g, 63 mmol) in ether (1400 mL) at −78° C. under nitrogen was added sequentially, dropwise, N,N,N′,N′-tetramethylenediamine (TMEDA, 9.45 mL, 63 mmol), and sec-butyllithium (53.3 mL, 69 mmol, 1.3 M solution in hexane). After 1 hr, freshly distilled compound 20 (10.1 g, 62.7 mmol) was added to the heterogenous mixture. The cooling bath was removed and the reaction mixture was allowed to warm to room temperature over 9 hr. Saturated NH₄Cl solution (400 mL) was then added and the mixture was stirred for 15 min. The ether layer was separated and the water layer was extracted with dichloromethane (4×100 mL). The organic layers were combined, dried (MgSO₄), and evaporated to a brown oil. The oil was dissolved in toluene (500 mL), and heated at reflux for 8 hr with 3.0 g of p-toluene sulfonic acid, cooled, and concentrated under vacuum. The residue was dissolved in dichloromethane, washed with dilute aqueous NaHCO₃, water, and then dried (Na₂SO₄), filtered, and evaporated to a gummy residue. On trituration with ethyl acetate/hexane (50:50), a solid precipitated. Recrystallization from ethyl acetate/hexane afforded 12.75 g (63%) of compound 22: mp 193-194° C.; IR (KBr) 1752 cm⁻¹ (C═O); ¹H NMR (CDCl₃) δ 2.47 (s, 3H, NCH₃), 2.88 (d, 1H, J=11.6 Hz), 3.02 (d, 1H, J=11.7 Hz), 3.76 (d, 1H, J=15.0Hz), 3.79 (d, 1H, J=15.1 Hz), 3.90 (s, 3H, OCH₃), 4.17 (s, 3H, OCH₃), 6.83 (d, H, J=8.4 Hz, ArH), 7.03 (d, 1H, J=8.2 Hz, ArH), 7.11 (m, 3H, ArH), 7.22 (m, 1H, ArH); MS (CI) m/z 326 (100).

2′,3′-Dihydro-4,5-dimethoxyspiro[isobenzofuran-1(3H),4′(1′H)-isoquinoline]-3-one (23). 1-chloroethyl chloroformate (5.1 mL, 46.3 mmol) was added dropwise to a suspension of compound 22 (6.21 g, 19.2 mmol) in 100 mL of 1,2-dichloroethane at 0° C. under nitrogen. The mixture was stirred for 15 min at 0° C., and then heated at reflux for 8 hr. The mixture was cooled, and concentrated under reduced pressure. To this mixture was added 75 mL of methanol and the reaction was heated at reflux overnight. After cooling, the solvent was evaporated to afford the hydrochloride salt of compound 23 in nearly quantitative yield. It was used in the next step without further purification: mp (HCl salt) 220-222° C.; mp (free base) 208-210° C.; IR (CH₂Cl₂, free base) 1754 cm⁻¹ (C═O); ¹H NMR (CDCl₃, free base) δ 3.18 (d, 1H, J=13.5 Hz), 3.30 (d, 1H, J=13.5 Hz), 3.84 (s, 3H, OCH₃), 3.96 (s, 3H, OCH₃), 4.02 (s, 2H, CH₂N), 6.67 (d, 1H, J=7.5 Hz, ArH), 7.12 (m, 2H, ArH), 7.19 (d, 1H, J=7.5 Hz, ArH), 7.26 (t, 1H, J=7.5 Hz, ArH), 7.41 (d, 1H, J=8.5 Hz, ArH); MS (CI) m/z 312 (100); HRCIMS Calc'd for C₁₈H₁₇NO₄: 312.1236; Found 312.1198; Anal. Calc'd for C₁₈H₁₇NO₄: C, 69.44. Found: C, 68.01.

2′,3′-Dihydro-4,5-dimethoxy-2′-p-toluenesulfonylspiro[isobenzofuran-1(3H),4′(1′H)isoquinoline]-3-one (24). Triethylamine (7 mL) was added dropwise to a mixture of p-toluenesulfonyl chloride (3.6 g, 18.9 mmole), compound 23 (as the HCl salt, obtained from 19.2 mmol of compound 22), and chloroform (100 mL) at 0 C. under nitrogen. After the addition was complete, the ice bath was removed and the reaction mixture was stirred at room temperature for 1 hr. It was then acidified with 100 mL of cold aqueous 0.1 N HCl, extracted with dichloromethane (2×100 mL), and the organic extract was dried (MgSO₄), filtered, and evaporated to afford a viscous liquid that on trituration with ethyl acetate/hexane at 0° C. gave a solid. Recrystallization from ethyl acetate/hexane afforded 8.74 g (97%, overall from compound 22) of compound 24: mp 208-210° C.; IR (KBr) 1767 cm⁻¹ (C═O); ¹H NMR (CDCl₃) δ 2.43 (s, 1H, CH₃), 3.22 (d, 1H, J=11 Hz), 3.88 (d, 1H, J=11 Hz), 3.90 (s, 3H, OCH₃), 3.96 (d, 1H, J=15 Hz), 4.17 (s, 3H, OCH₃), 4.81 (d, 1H, J=15 Hz), 6.97 (d, 1H, J=7.7 Hz, ArH), 7.16 (m, 3H, ArH), 7.26 (m, 1H, ArH), 7.38 (d, 2H, J=8 Hz, ArH), 7.72 (d, 2H, J=8 Hz, ArH); MS (CI) m/z 466 (100).

3,4-Dimethoxy-6-[(2-p-toluenesulfonyl-1,2,3,4-tetrahydroisoquinoline)-4-yl]benzoic acid (25). A solution of compound 24 (2.56 g, 5.51 mmol) in glacial acetic acid (250 mL) with 10% palladium on activated carbon (6.30 g) was shaken on a Parr hydrogenator at 50 psig for 48 hr at room temperature. The catalyst was removed by filtration, and the solvent was evaporated to afford 2.55 g (99%) of compound 25. An analytical sample was recrystallized from ethanol/water: mp 182-184° C.; IR (KBr) 1717 cm⁻¹ (COOH); ¹H NMR (DMSO-d₆) δ 2.35 (s, 3 H, CH₃), 3.12 (m, 1H), 3.51 (dd, 1H, J=5, 11.5 Hz), 3.71 (s, 6H, OCH₃), 4.10 (m, 1H, Ar₂CH), 4.23 (s, 2H, ArCH₂N), 6.52 (d, 1H, J=7.5 Hz, ArH), 6.78 (d, 1H, J=7.5 Hz, ArH), 6.90 (m, 1H, ArH), 7.07 (t, 1H, J=8 Hz, ArH), 7.14 (t, 1H, J=6.5 Hz, ArH), 7.20 (d, 1H, J=7.5 Hz, ArH), 7.38 (d, 2H, J=8 Hz, ArH), 7.63 (d, 2H, J=8.5 Hz, ArH); MS (CI) m/z 468 (16), 450 (63), 296 (100); HRCIMS Calc'd for C₂₅H₂₅NO₆S: 468.1481; Found: 468.1467.

2-N-p-Toluenesulfonyl-4-(2-hydroxymethyl-3,4-dimethoxyphenyl)-1,2,3,4-tetrahydroisoquinoline (26). To a solution of compound 25 (1.4 g, 2.99 mmol) in dry THF (30 mL) was added 1.0 M borane-tetrahydrofuran (8 mL) at 0° C. under N₂. After the addition was complete the mixture was stirred at reflux overnight. Additional borane-tetrahydrofuran (4 mL) was added and stirring was continued for another 30 min. After cooling and evaporating under reduced pressure, methanol (30 mL) was carefully added, and the solvent was removed at low pressure. The process was repeated three times to ensure the methanolysis of the intermediate borane complex. Evaporation of the solvent gave 1.10 g (81%) of compound 26. An analytical sample was purified by flash chromatography (silica gel, EtOAc/Hexane) followed by recrystallization from ethyl acetate/hexane: mp 162-164° C.; ¹H NMR (CDCl₃) δ 2.38 (s, 3H, CH₃), 3.18 (dd, 1H, J=7.5, 11.9 Hz), 3.67 (dd, 1H, J=4.5, 11.8 Hz), 3.81 (s, 3H, OCH₃), 3.85 (s, 3H, OCH₃), 4.27 (d, 1H, J=15 Hz), 4.40 (d, 1H, J=15 Hz), 4.57 (t, 1H, J=6 Hz, CHAr₂), 4.71 (s, 2H, CH₂OH), 6.58 (d, 1H, J=8.5 Hz, ArH), 6.74 (d, 1H, J=8.6 Hz, ArH), 6.84 (d, 1H, J=7.7 Hz, ArH), 7.08 (t, 2H, J=7.6 Hz, ArH), 7.14 (t, 1H, J=6.6 Hz, ArH), 7.27 (d, 2H, J=8 Hz, ArH), 7.65 (d, 2H, J=8 Hz, ArH); MS (CI) m/z 454 (2.57), 436 (100).

8,9-Dimethoxy-2-p-toluenesulfonyl-2,3,7,11b-tetrahydro-1H-napth[1,2,3-de]isoquinoline (27). Powdered compound 26 (427 mg, 0.98 mmol) was added in several portions to 50 mL of cold concentrated sulfuric acid (50 mL) at −40° C. under nitrogen with vigorous mechanical stirring. After the addition, the reaction mixture was warmed to −5° C. over 2 hr and then poured onto crushed ice (450 g) and left stirring for 1 hr. The product was extracted with dichloromethane (2×150 mL), washed with water (2×150 mL), dried (MgSO₄), filtered, and evaporated to afford an oil that on trituration with ether at 0° C. yielded compound 27 (353 mg, 82%) as a white solid that was used without further purification. An analytical sample was prepared by centrifugal rotary chromatography using 50% EtOAc/hexane as the eluent followed by recrystallization from EtOAc/hexane: mp 204-206° C.; ¹H NMR (CDCl₃) δ 2.40 (s, 3H, CH₃), 2.80 (m, 1H, H-1a), 3.50 (dd, 1H, J=4.5, 17.5 Hz, H-1b), 3.70 (dd, 1H, J=7, 14 Hz, H-3a), 3.828 (s, 3H, OCH₃), 3.832 (s, 3H, OCH₃), 3.9 (m, 1H, H-11b), 4.31 (d, 1H, J=17.6 Hz, H-7a), 4.74 (ddd, 1H, J=1.7, 6.0, 11.2 Hz, H-7b), 4.76 (d, 1H, J=14.8 Hz, H-3b), 6.77 (d, 1H, J=8.3 Hz, ArH), 6.87 (d, 1H, J=8.4 Hz, ArH), 6.94 (d, 1H, J=7.6 Hz, ArH), 7.13 (t, 1H, J=7.5 Hz, Ar—H-5), 7.18 (d, 1H, J=7.2 Hz, ArH), 7.33 (d, 2H, J=8.1 Hz, ArH), 7.78 (d, 2H, J=8.2 Hz, ArH); MS (CI) m/z 436 (55), 198 (86), 157 (100); HRCIMS Calc'd for C₂₅H₂₅NO₄S: 436.1583; Found: 436.1570.

8,9-Dimethoxy-2,3,7,11b-tetrahydro-1H-napth[1,2,3-de]isoquinoline (28). A mixture of compound 27 (440 mg, 1.01 mmol), dry methanol (10 mL) and disodium hydrogen phosphate (574 mg, 4.04 mmol) was stirred under nitrogen at room temperature. To this mixture was added 6.20 g of 6% Na/Hg in three portions and the reaction was heated at reflux for 2 hr. After cooling, water (200 mL) was added and the mixture was extracted with ether (3×200 mL). The ether layers were combined, dried (MgSO₄), filtered (Celite), and evaporated to give an oil that solidified under vacuum. After rotary chromatography 142 mg (50%) of compound 28 was obtained as an oil. The oil quickly darkened on exposure to air and was used immediately. A small portion of the oil was treated with ethereal HCl and the hydrochloride salt of compound 28 was recrystallized from ethanol/ether: mp (HCl salt) 190° C. (dec.); ¹H NMR (CDCl₃, free base) δ 3.13 (dd, 1H, J=10.8, 12 Hz, H-1a), 3.50 (dd, 1H, J=3.4, 17.4 Hz, H-1b), 3.70 (m, 1H, H-11b), 3.839 (s, 3H, OCH₃), 3.842 (s, 3H, OCH₃), 4.03 (dd, 1H, J=6, 12 Hz, H-7a), 4.08 (s, 2H, H-3), 4.33 (d, 1H, J=17.4 Hz, H-7b), 6.78 (d, 1H, J=8.24 Hz, ArH), 6.92 (m, 3H, ArH), 7.11 (t, 1H, J=7.5 Hz, ArH), 7.18 (d, 1H, J=7.5 Hz, ArH); MS (CI) m/z282 (100); HRCIMS Calc'd for C₁₈H₁₉NO₂: 282.1494; Found: 282.1497.

8,9-Dihydroxy-2,3,7,11b-tetrahydro-1H-napth[1,2,3-de]isoquinoline (29). To a solution of compound 28 (25 mg, 0.089 mmole) in dichloromethane (5 mL) at −78° C. was added boron tribromide (0.04 mL, 0.106 g, 0.42 mmol). After stirring at −78° C. under N₂ for 2 hr, the cooling bath was removed and the reaction mixture was left stirring at room temperature for 5 hr. It was then cooled to −78° C. and methanol (2 mL) was carefully added. After stirring for 15 min at room temperature, the solvent was evaporated. More methanol was added and the process was repeated three times. The resulting gray solid was recrystallized from ethanol/ethyl acetate to yield a total of 12 mg (41%) of the hydrobromide salt of compound 29: mp 258° C. (dec); ¹H NMR (HBr salt, CD₃OD) δ 3.43 (t, 1H, J=12 Hz, H-1a), 3.48 (dd, 1H, J=3.5, 18 Hz, H-1b), 4.04 (m, 1H, H-11b), 4.38 (dd, 2H, J=5.5, 12 Hz, H-7), 4.44 (s, 2H, H-3), 6.58 (d, 1H, J=8.5 Hz, ArH), 6.71 (d, 1H, J=8.5 Hz, ArH), 7.11 (d, 1H, J=7.5 Hz, ArH), 7.25 (t, 1H, J=7.5 Hz, ArH), 7.32 (d, 1H, J=7.5 Hz, ArH); MS (CI) m/z 254 (100); HRCIMS Calc'd for C₁₆Hl₅NO₂: 254.1181; Found: 254.1192.

### Example 11

### (R)-(+)-8,9-Dihydroxy-2,3,7,11b-tetrahydro-1H-napth[1,2,3-de]isoquinoline

5-Bromoisoquinoline. The apparatus was a 500 mL three-necked flask equipped with a condenser, dropping funnel, and a stirrer terminating in a stiff, crescent-shaped Teflon polytetrafluroethylene paddle. To the isoquinoline (57.6 g, 447 mmol) in the flask was added AlCl₃ (123 g, 920 mmol). The mixture was heated to 75-85° C. Bromine (48.0 g, 300 mmol) was added using a dropping funnel over a period of 4 hours. The resulting mixture was stirred for one hour at 75° C. The almost black mixture was poured into vigorously hand-stirred cracked ice. The cold mixture was treated with sodium hydroxide solution (10 N) to dissolve all the aluminum salts as sodium aluminate and the oily layer was extracted with ether. After being dried with Na₂SO₄ and concentrated, the ether extract was distilled at about 0.3 mm. A white solid (16.3 g, 78 mmol) from a fraction of about 125° C. was obtained (26% yield). The product was further purified by recrystallization (pentane or hexanes): mp 80-81° C.; ¹H NMR (DMSO-d₆) δ 9.34 (s, 1H), 8.63 (d, 1H, J=9.0Hz), 8.17 (d, 1H, J=7,5 Hz), 8.11 (d, 1H, J=6.6 Hz), 7.90 (d, 1H, J=6.0Hz), 7.60 (t, 1H, J=7.5 Hz); ¹³C NMR(DMSO-d₆) δ 153.0, 144.7, 134.3, 134.0, 129.3, 128.5, 128.0, 120.3, and 118.6. Anal. Calcd. for C₉H₆BrN: C, 51.96; H, 2.91; N, 6.73. Found: C, 51.82; H, 2.91; N, 6.64.

5-Isoquinolinecarboxaldehyde. To a solution of n-butyllithium (19.3 mL of 2.5 M in hexanes, 48 mmol) in a mixture of ether (80 mL) and THF (80 mL) at −78° C. was added dropwise a solution of bromoisoquinoline (5.0 g, 24 mmol) in THF (10 mL). The reaction mixture was stirred at −78° C. under argon for 30 minutes. Following the general procedures described by Pearson, et al., in J. Heterocycl. Chem., Vol. 6 (2), pp. 243-245 (1969), a solution of DMF (3.30 g, 45 mmol) in THF (10 mL) was cooled to −78° C. and quickly added to the isoquinolyllithium solution. The mixture was stirred at −78° C. for 15 minutes. Ethanol (20 mL) was added followed by saturated NH₄Cl solution. The resulting suspension was warmed to room temperature. The organic layer, combined with the ether extraction layer, was dried over Na₂SO₄. A pale yellow solid (2.4 g, 15 mmol, 64% yield) was obtained from chromatography (SiO₂ Type-H, 50% EtOAc in hexanes) and recrystallization (ethanol): mp 114-116° C.; ¹H NMR (DMSO-d₆) δ 10.40 (s, 1H), 9.44 (s, 1H), 8.85 (d, 1H, J=6.0Hz), 8.69(d, 1H, J=6.0Hz), 8.45 (m, 2H), 7.90 (t, 1H, J=7.2 Hz); ¹³C NMR (DM50-d₆) δ 194.23, 153.5, 146.2, 140.2, 135.2, 132.6, 130.2, 128.6, 127.5, and 117.2. Anal. Calcd. for C₁₀H₇NO.0.05 H₂O: C, 75.99; H, 4.53; N, 8.86. Found: C, 75.98, H, 4.66; N, 8.68.

α-(5-Bromo-1,3-benzodioxol-4-yl)-5-isoquinolinemethanol. To a solution of 4-bromo-1,2-(methylendioxy)benzene (3.01 g, 15 mmol) in THF (20 mL) at −78° C. was added dropwise lithium diisopropylamide (10.6 mL of 1.5 M in cyclohexane, 16 mmol). The reaction mixture was stirred at −78° C. under argon for 20 minutes. A brown solution was formed. A solution of 5-isoquinolinecarboxaldehyde (1.90 g, 12 mmol) in THF (4 mL) was added dropwise. The resulting mixture was stirred at −78° C. for 10 minutes and warmed to room temperature. Stirring was continued for 30 minutes at room temperature, and then the mixture was quenched with saturated NH₄Cl solution. The product was extracted with EtOAc and the solvent was removed under reduced pressure. Chromatography (SiO₂ Type-H, 35% EtOAc in Hexanes) of the residue yielded the title compound as a yellow solid (2.8 g, 7.8 mmol, 65% yield): mp 173-175° C.; ¹H NMR (DMSO-d₆) δ 9.32 (s, 1H), 8.47 (d, 1H, J=6.0 Hz), 8.05 (d, 1H, J=8.1 Hz), 7.96 (d, 1H, J=7.2 Hz), 7.76 (d, 1H, J=6.0 Hz), 7.66 (t, 1H, J=7.8 Hz), 7.14 (d, 1H, =8.1 Hz), 6.84 (d, 1H, J=8.1 Hz), 6.58 (d, 1H, J=8.1 Hz), 6.28 (d, 1H, J=5.4 Hz), 5.95 (s, 1H), 5.80 (s, 1H); ¹³C NMR (DMSO-d₆) δ 153.1, 147.6, 147.0, 142.9, 136.9, 132.7, 128.9, 128.3, 127.3, 126.7, 125.6, 124.4, 116.3, 114.0, 109.3, 101.6, and 69.0. Anal. Calcd. for C₁₇H₁₂BrNO₃: C, 57.01; H, 3.38; N, 3.91. Found: C, 57.04; H, 3.51; N, 3.89.

5-[(5-Bromo-1,3-benzodioxol-4-yl)methyl]isoquinoline. To a solution of secondary alcohol α-(5-bromo-1,3-benzodioxol-4-yl)-5-isoquinolinemethanol (8.37 mmol) in trifluoroacetic acid (100 mL), triethylsilane (83.7 mmol) was added and the resulting solution was refluxed for an hour at 70-75° C. and stirred overnight at room temperature. The solvent was removed under vacuum and the residue was dissolved in ethyl acetate, washed with saturated NH₄Cl dried over Na₂SO₄, filtered, and concentrated. Purification was performed by column chromatography to afford the trifluoroacetate salt of the title compound as a white crystalline solid (67% yield): mp 138-140° C.; ¹H NMR (CDCl₃) δ 9.64 (s, 1H), 8.63 (d, 1H, J=6.59 Hz), 8.45 (d, 1H, J=6.62 Hz), 8.14 (d, 1H, J=8.22 Hz), 7.77 (t, 1H, J=7.39 HZ), 7.64 (d, 1H, J=7.29 Hz), 7.13 (d, 1H, J=8.33 Hz), 6.71 (d, 1H, J=8.31 Hz), 5.94 (s, 2H), 4.53 (s, 2H); ³C NMR (CDCl₃) δ 147.8, 147.7, 147.1, 137.2, 135.1, 134.7, 133.4, 130.3, 128.6, 128.3, 125.9, 120.7, 119.4, 116.3, 109.1,101.2 and 31.7. Anal. Calcd. for C₁₇H₁₂BrNO₂.C₂HF₃O₂: C, 50.02; H, 2.87; Br, 17.51; N. 3.07. Found: C, 49.91; H, 3.02; Br, 17.95; N, 3.04.

Method A for 12H-Benzo[d,e][1,3]benzodioxol[4,5-h]isoquinoline. A solution of 5-[(5-bromo-1,3-benzodioxol-4-yl)methyl]-isoquinoline (0.357 g, 1.0 mmol) and 2,2′-azobisisobutylronitrile (0.064 g, 0.39 mmol) in benzene (10 mL) was cooled to −78° C., degassed four times with N₂ and then heated to 80° C. under argon. A solution of tributyltin hydride (1.14g, 3.9 mmol) in 10 mL of degassed benzene was added in two hours. TFA (0.185 g, 1.6 mmol) was added in four equal portions (¼ each half hour). The reaction mixture was stirred at 80° C. under argon for six hours after addition of TFA. Additional tributyltin hydride (0.228 g, 0.80 mmol) was added dropwise. The stirring continued overnight (16 hours). Another 2,2′-azobisisobutylronitrile (0.064 g, 0.39 mmol) and TFA (0.093 g, 0.80 mmol) were added in one portion. A solution of tributyltin hydride (1.14 g, 3.9 mmol) in 10 mL of degassed benzene was also added in two hours. More TFA (0.185 g, 1.6 mmol) was added in four equal portions (¼ each half hour). The stirring continued for another six hours and tributyltin hydride (0.456 g, 1.6 mmol) was added dropwise. The reaction mixture was stirred overnight (16 hours). The solvent was removed under reduced pressure. Pentane (100 mL) was added to the residue and the resulting mixture was cooled to −78° C. A brown gum was formed and filtered. The filtrate was extracted with MeCN. The MeCN layer was combined with the brown gum. The crude product from evaporation of MeCN was purified by chromatography (SiO₂ Type-H, 15% EtOAc in hexanes). The isolated compound was dissolved in Ch₂Cl₂ and extracted with HCl (1N). The aqueous layer was basified to pH˜10 using 10 N NaOH solution and reextracted with Ch₂Cl₂. The organic layer was dried over Na₂SO₄. Evaporation of solvent yielded the title compound as an orange solid (0.068 g, 0.26 mmol, 25% yield): mp 194-197° C.; ¹H NMR (DMSO-d₆) δ 9.12 (s, 1H), 9.06 (s, 1H), 7.93 (d, 1H, J=6.9 Hz), 7.83 (d, 1H, J=8.1 Hz), 7.73 (dd, 1H, J=7.2, 1.5 Hz), 7.66 (t, 1H, J=7.8 Hz), 6.96 (d, 1H, J=8.4 Hz), 6.14 (s, 2H), 4.44 (s, 2H); ¹³C NMR (DMSO-d6) 6 150.6, 147.0, 145.2, 135.6, 130.6, 129.3, 129.1, 127.7, 127.5, 125.0, 123.6, 117.2, 116.1, 107.5, 101.6, and 26.6. Anal. Calcd. for C₁₇H₁₁NO₂.0.12CH₂Cl₂: C, 75.75; H, 4.17; N, 5.16. Found: C, 75.75; H, 4.03; N, 4.83.

Method B. A solution of 5-[(5-bromo-1,3-benzodioxol-4-yl)methyl]-isoquinoline (12.6 g, 36.8 mmol) and 2,2′-azobisisobutylronitlile (5.92 g, 36.0 mmol) in benzene (1500 mL) was cooled to −78° C., degassed/purged four times with nitrogen and then heated to 80° C. under argon. A solution of tributyltin hydride (39.9 g, 137 mmol) in 30 mL of degassed benzene was added dropwise over a period of three hours. Acetic acid (12.5 g, 210 mmol) was added in one portion before the addition of tin hydride. The reaction mixture was stirred at 80° C. under argon for 16 hours. Excess triethylamine was added to neutralize the residual acetic acid component. The solvent was removed under reduced pressure. Methylene chloride (250 mL) was added to dissolve the semi-solid, followed by the addition of hexanes to a point just before the mixture became cloudy. This solution was poured over a short bed of silica gel and the tri-n-butyltin acetate was removed by washing with hexanes until no longer detected by TLC. The product was then eluted out with mixtures of hexanes and ethyl acetate to give the desired title compound (6.1 g, 23.4 mmol, 63.5% yield) which was identical to the product prepared by Method A.

Method A for (±)-8,9-Methylenedioxy-2,3,7,11b-tetrahydro-1H-napth[1,2,3-de]isoquinoline. To a solution of 12H-benzo[d,e][1,3]benzodioxol[4,5-h]isoquinoline (0.085 g, 0.33 mmol) in THF (43 mL) was added 2N HCl (1.7 mL, 3.4 mmol) and an orange precipitate formed. Sodium cyanoborohydride (0.274 g, 44 mmol) was added in one portion. The resulting suspension was stirred at room temperature for two hours. HCl (2N, 10 mL) was added and stirring continued for 5 minutes. Saturated NaHCO₃ solution was added (pH˜7-8). The resulting mixture was extracted with EtOAc, dried over Na₂SO₄ and the solvent was removed under reduced pressure. Chromatography (SiO₂ Type-H, 5% MeOH in CH₂Cl₂) of the residue yielded the title compound as a yellow gum (0.066 g, 0.25 mmol, 75% yield); ¹H NMR (CDCl₃) δ 7.15 (m, 2H), 6.97 (d, 1H, J=6.9 Hz), 6.83 (br, s, 1H), 6.68 (d, 1H, J=8.1 Hz), 6.59 (d, 1H, J=8.1 Hz), 6.01 (d, 1H, J=1.4 Hz), 5.91 (d, 1H, J=1.4 Hz), 4.40-4.00 (m, 5H), 3.55 (dd, 1H, J=17.7, 3.0 Hz), 3.10 (t, 1H, J=12.0 Hz); ¹³ C NMR (CDCl₃) δ 146.1, 144.8, 136.0, 132.2, 130.4, 128.6, 127.1, 127.0, 124.5, 118.5, 116.2, 106.2, 101.2, 45.8, 35.1, 34.3, and 28.9. Anal. Calcd. for C₁₇H₁₅NO₂.0.52HCN.1.8H₂O: C, 67.49; H, 6.18; N, 6.83. Found: C, 67.45; H, 5.96; N, 6.75.

Method B. 12H-Benzo[d,e] [1,3]benzodioxol[4,5-h]isoquinoline (11.26 g) was dissolved into 500 mL of glacial acetic acid in a suitable glass liner that will fit into a 1-L Parr “bomb reactor.” To this dark amber solution was added 480 mg PtO2 and a magnetic stirring bar. Usual purge cycles were repeated three times at −78° C. Finally hydrogen gas was charged into the steel bomb at 140 PSI while the content was still at −78° C. The reactor was allowed to warm to room temperature over a period of 2 hours while the internal pressure increased to 195 PSI. Gas absorption was faster after about 4 hours at room temperature. After 24 hours, the internal pressure returned to 165 PSI indicating roughly stoichiometric uptake of hydrogen gas. The black suspension was removed after the pressure was relieved, filtered over silica gel, rinsed with acetic acid, and concentrated under reduced pressure to give about 19 gm of gummy substance. The crude product was neutralized with sodium bicarbonate solution followed by extraction with methylene chloride to yield 11.6 gm of the title compound whose 1H NMR was indistinguishable from the purified material prepared above by the Method A.

(±)-8,9-Dihydroxy-2,3,7,11b-tetrahydro-1H-napth[1,2,3-de]isoquinoline. BBr₃ (25.0 mL of 1 M in CH₂Cl₂, 25.0 mmol) was added to a cooled solution (−78° C.) of methylenedioxy dinapsoline as prepared in Example 6 (1.4 g, 5.3 mmol) in Ch₂Cl₂. The mixture was stirred at −78° C. under nitrogen for three hours and then at room temperature overnight. After the mixture was cooled to −78° C., methanol (50 mL) was added dropwise and the solvent was removed by reduced pressure. The residue was dissolved in methanol (100 mL) and the solution was refluxed under nitrogen for 2 hours. After removal of solvent, chromatography (SiO₂, 10% MeOH in CH₂Cl₂) of the residue yielded the title compound as a dark brown solid (1.65 g, 4.94 mmol, 93% yield). MS (ESI) m/z 254 (MH⁺); ¹H NMR (DMSO-d₆) δ 9.50 (br, s, 2H), 9.28 (s, 1H), 8.54 (s, 1H), 7.32 (d, 1H, J=8.3 Hz); 7.23 (t, 1H, J=8.3 Hz), 7.12 (d, 1H, J=8.5 Hz), 6.70 (d, 1H, J=9.3 Hz), 6.54 (d, 1H, J=6.7 Hz), 4.37 (s 2H), 4.30-4.23 (m, 2H), 3.97 (m, 1 H), 3.45-3.31 (m, 2H); ¹³C NMR (DMSO-d6) δ 143.8, 142.0, 136.9, 132.1, 127.6, 127.0, 126.6, 124.1, 123.7, 114.0, 112.7, 46.6, 44.0, 32.9, and 28.5. Anal. Calcd. for C₁₆H₁₅NO₂.1.28HBr.0.59H₂O: C, 52.34; H, 4.79; N, 3.82. Found: C, 52.29; H, 4.92; N, 4:14.

R-(+)-8,9-Dihydroxy-2,3,7,11b-tetrahydro-1H-napth[1,2,3-de]isoquinoline. Step A. (+)-8,9-Methylenedioxy-2,3,7,11b-tetrahydro-1H-napth[1,2,3-de]isoquinoline. A sample of racemic (±)-8,9-methylenedioxy-2,3,7,11b-tetrahydro-1H-napth[1,2,3-de]isoquinoline was injected into a preparative HPLC (Dynamax Rainin Model SD-1) equipped with Chiralcel OD column (5 cm×50 cm, 20μ, Chiral Technologies, Inc) at a flow rate of 50 mL/min using UV detector set at λ=220 nm. Using an isocratic method, the solvent system (5% Ethanol/Hexanes, 0.1% TFA) was found to best separate the enantiomers. As much as 150 mg/5mL ethanol can be injected to the column per run. A total of 425 mg of racemic (±)-8,9-methylenedioxy-2,3,7,11b-tetrahydro-1H-napth[1,2,3-de]isoquinoline injected can produce about 200 mg of each enantiomer. Optical rotation was taken for each of the enantiomer collected: 1^{st} Peak (Rf=19.6 minutes): [α]_{D} −88.9° (c 0.03, CHCl₃); 2^{nd} Peak (Rf=23.6 minutes); [α]_{D} −90.3° (c 0.03, CHCl₃).

One of these two isomers was derivatized into the corresponding N-(p-tolylsulfonamide) for a single crystal X-ray determination. From there it was concluded that the chirality of the (−)-isomer of Formula VIIb has (S)-configuration at the asymmetric center. The second peak is the desired title compound.

Step B. R-(+)-8,9-Dihydroxy-2,3,7,11b-tetrahydro-1H-napth[1,2,3-de]isoquinoline. Using the identical deprotection procedure described for the racemic compound in Example 7, each of these isomers were subjected to BBr₃ deprotection to give chiral (+) and (−)-isomers of dinapsolines (DNS).

| | DNS from first peak | DNS from second peak |
|---|---|---|
| Optical | −70.7° (c 0.03, MeOH) | +75.0° (c 0.03, MeOH) |
| rotations [α]_{D} | | |

(R)-(+)-8,9-Dihydroxy-2,3,7,11b-tetrahydro-1H-napth[1,2,3-de]isoquinoline. Step A. (±)-8,9-Methylenedioxy-2,3,7,11b-tetrahydro-1H-napth[1,2,3-de]isoquinoline A solution of racemic (±)-8,9-methylenedioxy-2,3,7,11b-tetrahydro-1H-napth[1,2,3-de]isoquinoline (3.0 gm, 11.3 mmol) in 100 mL of 95% ethyl alcohol at room temperature was mixed with a warm solution of (+)-dibenzoyl-D-tartaric acid in 40 mL of 95% ethyl alcohol. The solution was allowed to stand at room temperature for 4 hours and the grayish off-white crystals were collected by filtration and subsequently dried in a vacuum oven at 35° C. to give 1.3 gm (melting point: 175-176° C., 35.7%). The enantiomeric purity was determined by the same chiral HPLC conditions described above in Example 8: the salt was neutralized with 2M potassium hydroxide solution and the organic materials extracted with methylene chloride. The organic layers were combined and concentrated under reduced pressure to give a white solid which was redissolved in methanol prior to injection into HPLC Chiral column. The ratio of the second peak to the first was determined to be greater than 40:1. The identical resolution may also be carried out using the unnatural D-tartaric acid. Melting points are uncorrected for the desired tartaric salts of the title compound. (R)-(+)-(+)-8,9-methylenedioxy-2,3,7,11b-tetrahydro-1H-napth[1,2,3-de]isoquinoline (+)-dibenzoyl-D-tartaric acid salt: mp 175-176° C. (R)-(+)-(+)-8,9-methylenedioxy-2,3,7,11b-tetrahydro-1H-napth[1,2,3-de]isoquinoline D-tartaric acid salt: mp 186-188° C.; [α]²⁵=+90.3°.

Step B. (R)-(+)-8,9-dihydroxy-2,3,7,11b-tetrahydro-1H-napth[1,2,3-de]isoquinoline. The free base is regenerated from the tartaric salts by neutralization. The (+)-isomer of dinapsoline prepared by deprotection as described in Example 7 is identical to the (+)-isomer of Example 8.

### Example 12

### Asymmetric Synthesis of Optically Active (+)-DHX.

Asymmeteric syntheses of DHX are generally described in Tomioka et al. (2001) “Electronic and steric control in regioselective addition reactions of organolithium reagents with enaldimines” J. Org. Chem., 66:7051-7054; Tomioka et al. (2001) “Molecular assembly and gelating behavior of didodecanoylamides of alpha,omega-alkylidenediarnines” J. Am. Chem. Soc., 123:11817-11818; Yamashita et al. (2004) “Construction of arene-fused-piperidine motifs by asymmetric addition of 2-trityloxymethylaryllithiums to nitroalkenes: the asymmetric synthesis of a dopamine D1 full agonist, A-86929” J. Am. Chem. Soc., 126:1954-1955. The first synthesis begins with unsaturated ester 7, which is prepared from the corresponding acid and 2,6-di-t-butyl-4-methoxyphenol in the presence of trifluoroacetic anhydride as shown in FIG. 5. The key step of the route takes advantage of a chiral ether-directed asymmetric conjugate addition of phenyllithium to 7, giving ester 8 (93%). It is necessary to use the sterically hindered ester to prevent attack of the aryllithium on the ester carbonyl. Transformation of the cis product 8 to the requisite trans acid 9 is readily accomplished by treatment with sodium methoxide, followed by sodium hydroxide. Acid 9 produced in this manner was determined to have 74% ee.

Enantiopurification of 9 occurs upon recrystallization of its dicyclohexylamine salt in ethanol. A Curtius rearrangement and protection of the incipient amine affords compound 10, which is then subjected to a Bischler-Napieralski cyclization providing the core benzophenanthridine nucleus. Removal of both the nitrogen and the catechol oxygen protecting groups completes the synthesis of (+)-DHX.

### Method Example 1

### Binding Affinity at Rat Brain Striatal D₁ and D₂ Receptors

The affinity of the compounds described in Examples 2, 3, 5, and of dihydrexidine (Example 1) for D₁ and D₂ receptors was assayed utilizing rat brain striatal homogenates having D₁ and D₂ receptors labeled with ³H-SCH 23390 and ³H-spiperone, respectively. The results were compared to dopamine and the agonists chlorpromazine and SCH 23390. The data obtained are shown in Table II.

TABLE II

| | D₁ | D₂ | D₁:D₂ |
|---|---|---|---|
| Compound | Affinity ^{(a)} | Affinity ^{(a)} | Selectivity |
| 6b | 14 | 650 | 46 |
| 6c | 7 | 45 | 6 |
| 6e | 290 | 185 | 0.6 |
| Dihydrexidine (6a) | 5.5 ± 0.9 | 61.0 ± 5.0 | 13 |
| Dopamine | 500 | 70 | 0.1 |
| Chlorpromazine | — | 1.2 ± 0.3 | — |
| SCH 23390 | 0.69 ± 0.05 | — | — |

^{(a)} Affinity (K_{0.5} in nM) ± SEM.

### Method Example 2

### Binding and Activity of Dihydrexidine at Dopamine Receptors

| | | | Cloned Receptors (nM) | | | | |
|---|---|---|---|---|---|---|---|
| | | | D_{1A} | D_{2L} | D₃ | D₄ | D₅ |
| | Rat Striatum (nM) | | C-6 | C-6 | C-6 | CHO | HEK |
| Drug | D₁-like | D₂-like | (monkey) | (rat) | (rat) | (rat) | (human) |
| SCH 23390 | 0.69 | — | 0.32 | — | — | — | 1.0 |
| chlorpromazine | — | 1.19 | — | 0.74 | 0.9 | 20 | — |
| dihydrexidine | 5.5 | 24.4 | 2.2 | 183 | 18 | 13 | 16 |

Dihydrexidine was screened for activity at 40 binding sites (other than the D₁ site) and been found to be inactive (IC₅₀>10 μM) at all except D₂ dopamine receptors IC₅₀=130 nM) and alpha₂ adrenergic receptors (IC₅₀=ca. 230 nM). Aside from the D₁ site, dihydrexidine appears to stimulate only postsynaptic D₂ dopamine receptors. Dihydrexidine is as efficacious and is approximately 70 times more potent than dopamine in the stimulation of adenylate cyclase. This effect is blocked by the D₁ antagonist SCH 23390, but not by D₂5-HT₂, muscarinic, or alpha- or beta-adrenergic receptor antagonists. Dihydrexidine shows full efficacy in stimulating adenylate cyclase in rat, monkey, and human brain tissue. Dihydrexidine is inactive in releasing dopamine or in blocking its reuptake.

### Method Example 3

### Binding and Activity of Dinapsoline at Dopamine Receptors

| | | | Cloned Receptors (nM) | | | | |
|---|---|---|---|---|---|---|---|
| | | | D_{1A} | D_{2L} | D₃ | D₄ | D₅ |
| | Rat Striatum (nM) | | C-6 | C-6 | C-6 | CHO | HEK |
| Drug | D₁-like | D₂-like | (monkey) | (rat) | (rat) | (rat) | (human) |
| SCH 23390 | 0.69 | — | 0.32 | — | — | — | 1.0 |
| chlorpromazine | — | 1.19 | — | 0.74 | 0.9 | 20 | — |
| dinapsoline | 5.93 | 31.3 | 6.1 | 59 | 10 | 60 | 5.0 |
| SKF 38393 | 20 | — | 8.6 | — | — | — | 80 |
| quinpirole | >5000 | 28.8 | — | 221 | 4.5 | — | — |

Dinapsoline was as effective as dopamine in activating adenylate cyclase in rat brain striatum. In addition, dinapsoline was as effective as dopamine even when receptor reserve is reduced, indicating equal intrinsic activity.

Dinapsoline also displayed full agonist activity in stimulating adenylate cyclase (AC) at the cloned human D₁-like receptors. Dinapsoline is equally efficacious and more potent at both the D₁ and D₅ receptors when compared to dopamine. The data for several experiments are summarized in the following table, indicating that dinapsoline does not functionally discriminate between the D₁ and D₅ receptors for stimulating AC:

Dinapsoline potently activates hD₁ and hD₅ receptors

EC50 (nM) ± SEM

| Test Ligand | D₁ | D₅ |
|---|---|---|
| dopamine | 486 ± 157 | 114 ± 186 |
| dinapsoline | 28 ± 9 | 10 ± 2 |

Studies completed in HEK cells represent at least three separate experiments (expressed as mean±SEM).

### Method Example 4

### Pulmonary Delivery System

The AERx delivery system (Aradigm Corporation, Hayward, Calif.) may be used with the compounds and compositions described herein for delivery according to the methods described herein. See generally, Okumu et al. (2002) “Evaluation of the AERx pulmonary delivery system for systemic delivery of a poorly soluble selective D-1 agonist, ABT-431” Pharm. Res., 19:1009-1012.; Zheng et al. (1999) “Pulmonary delivery of a dopamine D-1 agonist, ABT-431, in dogs and humans” Int. J. Pharm., 191:131-140.

The central element of the AERx system is the AERx Strip dosage form, which contains a 50-μL blister for the liquid formulation and a disposable nozzle delivery. The nozzle's design can be adjusted for various formulation characteristics and treatment requirements to regulate the particle size and thus the primary deposition area of the therapy. The AERx devices utilize a piston mechanism to expel formulation from the AERx Strip with high efficiency. Several versions of the AERx platform have been developed. One version is an electromechanical AERx device. The corresponding all-mechanical device is the AERx ESSENCE.

### Method Example 5

### Formulations

Formulations include high concentration liquid formulations and powder formulations. The compounds described herein may be prepared in a lyophilized form, which may be pre-filled into a portable AERx device as described herein. The dry power may be reconstituted for pulmonary administration. In one aspect, the particle size is in the range from about 2 to about 3 μm for delivery to the alveolar region in the lungs. Alternatively, high liquid concentration formulations may be delivered using other conventional atomizing or aerosolizing devices. In one aspect, the liquid concentration is at least 50%, in another aspect, the liquid concentration is at least 60%, and in another aspect, the liquid concentration is at least 70%. It is appreciated that the liquid volume concentration may be increased by modifying pH, ionic strength, and other solute properties.

### Method Example 6

### Injured Lung Model in Rat

The compounds described herein are assayed for effectiveness for lung liquid clearance in a rat model of lung injury by. Mechanical ventilation is utilized to induce lung injury. Pharmacological antagonists are used to validate the involvement of the D₁ receptor in lung edema clearance. The effect observed may be compared to that observed with dopamine and isoproterenol as standards.

The assay is generally described in Lecuona et al. (1999) “Ventilator-associated lung injury decreases lung ability to clear edema in rats” Am. J. Respir. Crit Care Med., 159:603-609; Saldias et al. (2002) “Dopamine increases lung liquid clearance during mechanical ventilation” Am. J. Physiol Lung Cell Mol. Physiol, 283:L136-L143; Saldias et al. (2000) “beta-adrenergic stimulation restores rat lung ability to clear edema in ventilator associated lung injury” Am. J. Respir. Crit Care Med., 162:282-287. These studies evaluate the effect of administration of compounds described herein in rats exposed to high tidal volumes (to simulate lung injury). Rats are exposed to high (induce lung injury) or low (control for exposure to ventilation) tidal volumes. These studies determine the effect of compound administration on alveolar fluid resorption in three groups, control, low tidal volume, and high tidal volumes. It is appreciated that dopamine may be used as a positive control.

Mechanical ventilation with high tidal volumes (HVT) has been shown to cause or worsen lung injury, as well as to adversely affect lung function. HVT causes mild to moderate lung injury in rats, decreasing alveolar fluid resorption by ˜50% compared to control rats, an effect not seen when rats are exposed to low volume titers (LVTs). Dopamine administration into the airspace causes a significant increase in lung liquid clearance, rescuing HVT-exposed animals to the same clearance level as control and LVT-treated animals. Adult rats are anesthetized with 50 mg of pentobarbital/kg body weight given intraperitoneally (ip), tracheotomized, and mechanically ventilated using a rat ventilator (model 683; Harvard Apparatus, South Natick, Mass.). HVT is defined as ventilation for 40 min at 40 mL/kg, peak airway pressure of 35 cm H₂O, and respiratory rate (RR) of 40 breaths/min without positive end-expiratory pressure. LVT animals will also be ventilated for 40 min, though under the following conditions: tidal volume of ˜10 mL/kg, peak airway pressure of 8 cm H₂O, RR of 40 breaths/min. The control group does not receive ventilation.

Prior to and during mechanical ventilation, the rats are anesthetized. Pathogen-free, male, Sprague-Dawley rats (300-325 g) are anesthetized via intraperitoneal injection of Ketamine (60-80 mg/kg/body weight)/Xylazine (5-10 mg/kg/body weight). A tuberculin syringe (1 cc 26 G ⅜ syringe and needle) is used to administer the anesthesia. Complete anesthesia is assessed by observation of ear and toe pinch reflexes. The trachea is cannulated with a 16G catheter. Rats are ventilated with a rodent ventilator with 100% Oxygen (Harvard apparatus, model 683) for 40 min using the following experimental protocols: (A) Low tidal volume (LVT): tidal volume of 10 ml/kg and peak airway pressure of ˜8 cm H₂O. (B) High tidal volume (HVT): tidal volume of 40 ml/kg and peak airway pressure of ˜35 cm H₂O and compared to (C) control non-ventilated rats. Immediately following Mechanical Ventilation Induced Lung Injury Model, the rats will be used in the Isolated Perfused Rat Lung Model.

### Method Example 7

### Isolated Perfused Rat Lung Model

Pathogen-free, male, Sprague-Dawley rats (300-325 g) are anesthetized with sodium pentobarbital (Nembutal) (40 mg/kg body weight, I.P.). Complete anesthesia is assessed by observation of ear and toe pinch reflexes. The trachea is cannulated with a 16G catheter. Animals are placed on a Harvard small rodent ventilator with 100% oxygen for ˜10 min at 5 cm H₂0 airway pressure. A midline laprotomy and thoracotomy will be performed. Animals will be exsanguinated by laceration of the renal artery. The lungs and heart will then be removed en bloc. Total surgical time, from skin incision through removal of the lungs, is about 10 to 15 minutes. The pulmonary artery and left atrial appendage are cannulated and perfused with a solution of 3% bovine serum albumin (BSA) in buffered physiological salt solution.

The three groups of animals (control, LVT and HVT) are then tested in the rat isolated perfused model. The general method is to measure lung liquid clearance, which is described in Azzam et al. (2001) “Catecholamines increase lung edema clearance in rats with increased left atrial pressure” J. Appl. Physiol, 90:1088-1094.4; and elsewhere. Lungs are isolated from anesthetized rats after exposure to a 10-min ventilation with 100% O₂. This exposure to O₂ facilitates the filling of the alveoli with instillate fluid. Both the pulmonary artery and left atrial appendage are cannulated and perfused with a solution containing 3% bovine serum albumin in buffered physiological salt solution. Fluorescein-labeled (FITC)-albumin in the perfusate in order to monitor the leakage of protein from the vascular space into the airways. Generally this step provides a quantitative method for measuring fluid clearance. The recirculating volume of a constant-pressure perfusion system will be set at 90 mL, with arterial and venous pressures set at 12 and 0 cm H₂O, respectively. The lungs are excised from the thoracic cavity and placed in a ‘pleural’ bath containing 100 mL of the BSA solution. The system is maintained at a constant temperature of 37° C. in a water bath.

The lungs are then instilled with 5 mL BSA containing Evans blue dye-labeled (EBD)-albumin, 22Na+, and 3H-mannitol through a tracheal catheter. Generally, this step provides a quantitative method for measuring fluid clearance. Following a 10 min equilibration period, samples are taken at 0 and 60 min from the instillate, the perfusate, and the bath solution. The samples are centrifuged and then EBD-albumin is measured by absorbance at 620 nm, FITC-albumin by fluorescence (excitation at 487 nm and emission at 520 nm), and 22Na+ and 3H-mannitol by scintillation counting. The EBD-albumin allows the determination of the fluid volume that remains in the lung, as it remains in the airspace. The decrease in 22Na+ and 3H-mannitol in the instillate indicates the total and passive small solute and fluid movement. 22Na+ and 3H-mannitol will be purchased from DuPont NEN (Boston, Mass.).

This experimental set up allows the investigation of the administration of the compounds described herein in two different manners, by the alveolar instillate or perfused through the pulmonary vasculature. Previous research has demonstrated that whereas administration of dopamine in the perfusate does increase lung liquid clearance, this effect is not as consistent as that observed when dopamine is administered through addition to the instillate. Without being boudn by heory, the difference may be due to the concentration of dopamine at the epithelial cell level following instilled delivery. Therefore, experiments using instilled administration may be superior. Six concentrations of the compounds described herein (10-11, 10-10, 10-9, 10-8.5, 10-7 and 10-6 M) or four concentrations of dopamine (10-10, 10-9, 10-8 and 10-6 M) are administered to rats. Each concentration is evaluated in eight rats.

In order to demonstrate that the effect observed after compound and dopamine administration is due to activation of the D₁ receptor, the effect of administration of DAR-O1OOA and dopamine in conjunction with the D₁ antagonist SCH23390 is tested. SCH23390 (100 μM) is administered to eight rats with either dopamine (10-6) or DAR-O1OOA (10-7). A group of eight rats that are not exposed to compounds or dopamine serves as the control. The compounds may be prepared fresh daily in normal saline as 1000× stocks.

The details for the calculation as well as the equations needed for determining edema clearance are outlined in Saldias et al. (1999) “Dopamine restores lung ability to clear edema in rats exposed to hyperoxia” Am. J. Respir. Crit Care Med., 159:626-633. Following calculation of fluid clearance, all data is expressed as mean±SEM and subjected to ANOVA to test for differences between groups, followed by a multiple comparison test (Tukey) when the F statistic indicates significance. Groups that receive the compounds described herein are compared to those that receive dopamine for potency and efficacy.

### Method Example 8.

### SARS Pneumonia Model in Ferret

Castrated and descented male fitch ferrets weighing between 800-1200 grams are purchased from Marshall Farms USA, Inc (North Rose, N.Y.) and are pair-housed in an AAALAC-accredited facility (Registration #000643). Animals are maintained on a 12-hour dark/light schedule. Animals are provided food and water ad libitum. All procedures are in accordance with the NRC Guide for the Care and Use of Laboratory Animals, the Animal Welfare Act, and the CDC.NIH Biosafety in Microbiological and Biomedical Laboratories. In addition, all procedures are approved by the Southern Research Institute Institutional Animal Care and Use Committee and the Southern Research Institute Institutional Biosafety Committee.

The “Toronto-2” (Tor2) HCoV-SARS strain was provided by Dr. Heinz Feldmann from the Canadian Science Centre for Human and Animal Health, Winnipeg, Canada (Health Canada). The virus was isolated from a fatal Canadian SARS case and passaged twice in VeroE6 cells at Health Canada. The virus was passaged once in VeroE6 cells to generate the virus stock, to a titer of ˜2.1×108 PFU/ml by standard plaque assay. Animals are observed for clinical signs daily throughout the study period. Blood withdrawals are performed for functional immunology studies.

A number of antiviral drug candidates may be used in conjunction with the compounds described herein for their ability to decrease viral infection and pulmonary edema. Animals will be challenged with the Toronto-2 (Tor2) strain of HCoV-SARS by intranasal installation (106 PFU in PBS). Animals will be administered with sponsor-provided material via intratracheal route as outlined. For intratrachael administration, ferrets are anesthetized with ketamine (25 mg/kg), xylazine (2 mg/kg), and atropine (0.05 mg/kg) by the intramuscular route and vaccinated. Blood is collected from ferrets via the anterior vena cava or subclavial vein prior to study and at scheduled times post treatment. The first experiment involves the an antiviral drug and a compound described herein, each at three broad concentrations. This experiment defines the working concentration of test compound used in conjunction with the antiviral drug. A second experiment is performed with a more narrow range of the test compound concentration.

During the study the ferrets are monitored daily for changes in temperature, weight gain, and clinical signs. Clinical signs of sneezing (before anesthesia), inappetance, dyspnea, and level of activity are assessed daily. A scoring system is used to assess activity level where 0=alert and playful; 1=alert but playful only when stimulated; 2=alert but not playful when stimulated; 3=neither alert nor playful when stimulated. Based on the daily scores for each animal in a group, a relative inactivity index is calculated as _(day 1 to day 7) [Score+1]n/_(day 1 to day 7) n, where n equals the total number of observations. A value of 1 is added to each base score so that a score of “0” could be divided by a denominator, resulting in an index value of 1.0. Two and five days after infection, four (4) animals are sacrificed and tissues assayed for viral load (by plaque assay, TCID50 and RT-PCR) as well as histopathology/immunology as described below.

Blood is withdrawn from each animal at 24 hours prior to study onset and prior to sacrifice on day 7. Blood is centrifuged and serum collected and stored at −20° C. until used. Serum samples are used for ELISA using standard procedures.

Tissues are aseptically harvested following Tor2 challenge (spleen and lungs) after humane euthanasia (pentobarbital for ferrets). Animals are sacrificed at the time point of peak viral replication in lungs as previously determined; day 2 for ferrets. Animals are sacrificed and tissues harvested at the time point of optimal pathology as previously determined; day 5 for ferrets.

Ferret lungs are used for virus titration and pathology. Lungs are weighed and examined for gross pathology and photographs are taken using a digital camera. One lobe from each animal is fixed in formalin for sectioning (described later). Remaining lobes will be lavaged using standard procedures and bronchial-alveolar lavage (BAL) fluid and cells collected. BAL is centrifuged to collect cells (pellet) and lavage fluid used for virus titration/PCR.

BAL is split into equal fractions and one-half treated with Trizol reagent for RNA isolation and the other half used for virus titration by TCID50 assay and plaque assay (following TCID50 on specific samples). Trizoltreated supernatant fluid is examined for the presence of HCoV-SARS N-protein MRNA using standard RT-PCR methods previously described. BAL analysis includes the Cell pellet from above which is resuspended in media and spun onto slides using a cytocentrifuge. Duplicate slides are stained with hematoxylin and eosin and surface marker-specific antibodies using standard procedures. The results are presented as cell type and number.

Ferret lungs are examined at day +2 and +5 post infection using standard procedures. Briefly, lungs are fixed in 10% neutral buffered formalin (24 hr) and embedded into paraffin. Tissue sections of 5 μm are prepared on electrostatically charged glass slides and then baked at 60° C. Sections are stained by hematoxylin and eosin and/or probed with anti-SARS antibody and visualized using microscopy. Photomicrographs and pathology report are provided for each study group.

The foregoing exemplary compounds, synthetic procedures, formulations, delivery devices, etc. are intended to be illustrative of the invention described herein and it is to be understood that such exemplary compounds and synthetic procedures are not to be construed as limiting the invention in any way. Variations of the foregoing are therefore contemplated herein, such as for example, prodrugs of the compounds described herein where the compounds include a vicinal pair of aromatic hydroxyl groups. In those variations, both hydroxyl groups may be protected or otherwise derivatized to include active ester groups that may be the same or different. In other variations, only one of the hydroxyl groups is protected or otherwise derivatized to include an active ester group. Illustrative examples of such variations are described in U.S. patent application Ser. No. 10/503,796, the described compounds and synthetic procedures of which are incorporated herein by reference.

What is claimed is:

1. A method for treating a patient having pulmonary edema, said method comprising the step of administering to the lung endobronchial space of the airways of said patient an effective amount of a dopamine D₁ receptor full agonist, where the dopamine receptor agonist is a compound selected from the group consisting of hexahydrobenzophenanthridines, hexahydrothienophenanthridines, phenyltetrahydrobenzazepine, chromenoisoquinolines, and naphthoisoquinolines, combinations thereof, and pharmaceutically acceptable salts thereof, and where the dopamine D₁ receptor agonist is a full agonist, and where the dopamine receptor agonist is in the form of an aerosol or a dry powder.

2. (canceled)

3. The method of claim 1 wherein the dopamine D₁ receptor agonist is more active at dopamine D₁ receptors than dopamine D₂ receptors.

4. (canceled)

5. (canceled)

6. (canceled)

7. (canceled)

8. A method for treating a patient having pulmonary edema, said method comprising the step of administering to the lung endobronchial space of the airways of said patient an effective amount of a dopamine D₁ receptor full agonist, where the dopamine receptor agonist is a compound selected from the group consisting of hexahydrobenzophenanthridines, chromenoisoquinolines, and naphthoisoquinolines, and pharmaceutically acceptable salts thereof, and where the dopamine receptor agonist is in the form of an aerosol or a dry powder.

9. (canceled)

10. (canceled)

11. The method of claim 8 wherein the dopamine D₁ receptor agonist is more active at dopamine D₁ receptors than dopamine D₂ receptors.

12. (canceled)

13. (canceled)

14. The method of claim 8 wherein the dopamine D₁ receptor agonist has a plasma half-life of less than about 6 hours.

15. (canceled)

16. (canceled)

17. (canceled)

18. (canceled)

19. The method of claim 8 wherein the dopamine D₁ receptor agonist is a compound of the formula: 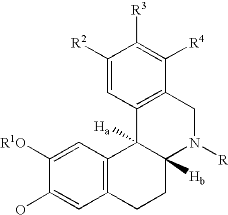
or a pharmaceutically acceptable salt thereof, wherein:
Hₐ and H_{b} are trans across the ring fusion bond;
R is hydrogen or C₁-C₄ alkyl;
R¹ is hydrogen, C₁-C₄ alkyl, acyl, an active ester group, or an optionally substituted phenyl protecting group;
X is hydrogen, or a group —OR⁵ wherein R⁵ is hydrogen, C₁-C₄ alkyl, benzoyl, pivaloyl, an active ester group, or an optionally substituted phenyl protecting group; or when X is the group —OR⁵, R¹ and R⁵ are taken together to form a divalent radical selected from the group consisting of —CH₂— and —(CH₂)₂—;
R², R³, and R⁴ are each independently selected from the group consisting of hydrogen, C₁-C₄ alkyl, phenyl, fluoro, chloro, bromo, iodo, and a group —OR⁶ wherein R⁶ is hydrogen, benzoyl, pivaloyl, or an optionally substituted phenyl protecting group.

20. The method of claim 19 wherein at least one of the groups R², R³, and R⁴ is methyl.

21. The method of claim 19 wherein X is hydroxy.

22. The method of claim 19 wherein R is hydrogen.

23. The method of claim 19 wherein R is C₁-C₄ alkyl.

24. (canceled)

25. (canceled)

26. (canceled)

27. (canceled)

28. (canceled)

29. The method of claim 8 wherein the dopamine D₁ receptor agonist is a compound of the formula: 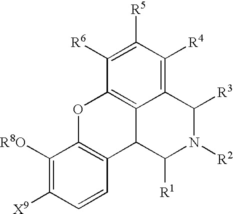
or a pharmaceutically acceptable salt thereof, wherein:
R¹, R², and R³ are each independently selected from the group consisting of hydrogen, C₁-C₄ alkyl or C₂-C₄ alkenyl;
R⁴, R⁵, and R⁶ are each independently selected from the group consisting of hydrogen, C₁-C₄ alkyl, phenyl, halogen, or a group having the formula —OR, where R is as defined above;
R⁸ is hydrogen, C₁-C₄ alkyl, acyl, an active ester group, or an optionally substituted phenyl protecting group;
X⁹ is hydrogen, or a group —OR⁹ wherein R⁹ is hydrogen, C₁-C₄ alkyl, benzoyl, pivaloyl, an active ester group, or an optionally substituted phenyl protecting group; or when X is the group —OR⁹, R⁸ and R⁹ are taken together to form a divalent radical selected from the group consisting of —CH₂— and —(CH₂)₂—.

30. The method of claim 8 wherein the dopamine D₁ receptor agonist is a compound of the formula: 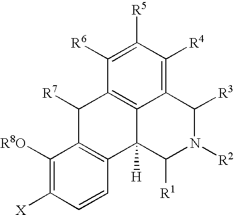
and pharmaceutically acceptable salts thereof, wherein
R¹, R², and R³ are each independently selected from the group consisting of hydrogen, C₁-C₄ alkyl, and C₂-C₄ alkenyl;
R⁴, R⁵, and R⁶ are each independently selected from the group consisting of hydrogen, C₁-C₄ alkyl, phenyl, halogen, and a group having the formula —OR, where R is hydrogen, acyl, an active ester group, or an optionally substituted phenyl protecting group;
R⁷ is selected from the group consisting of hydrogen, hydroxy, C₁-C₄ alkyl, C₂-C₄ alkenyl, C₁-C₄ alkoxy, and C₁-C₄ alkylthio;
R⁸ is hydrogen, acyl, an active ester group, or an optionally substituted phenyl protecting group; and
X is hydrogen, or a group —OR⁹ wherein R⁹ is hydrogen, benzoyl, pivaloyl, an active ester group, or an optionally substituted phenyl protecting group; or when X is the group —OR⁹, R⁸ and R⁹ are taken together to form a divalent radical selected from the group consisting of —CH₂— and —(CH₂)₂—.

31. A pharmaceutical composition comprising a compound and a pharmaceutically acceptable carrier therefor, where the compound and the carrier are adapted for delivery into the lung endobronchial space of the airway of a patient in need of relief from pulmonary edema in a dry powder or liquid concentrate formulation, wherein the compound is selected from the group consisting of hexahydrobenzophenanthridine, hexahydrothienophenanthridine, phenyltetrahydrobenzazepine, chromenoisoquinoline, and naphthoisoquinoline dopamine D₁ receptor agonists, and where the compound is present in the composition in an amount effective to treat the pulmonary edema of said patient.

32. (canceled)

33. (canceled)

34. (canceled)

35. The pharmaceutical composition of claim 30 wherein the compound is a compound of the formula: 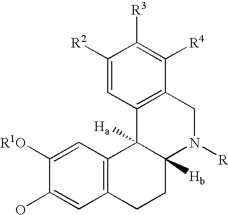
or a pharmaceutically acceptable salt thereof, wherein:
Hₐ and H_{b} are trans across the ring fusion bond;
R is hydrogen or C₁-C₄ alkyl;
R¹ is hydrogen, C₁-C₄ alkyl, acyl, an active ester group, or an optionally substituted phenyl protecting group;
X is hydrogen, or a group —OR⁵ wherein R⁵ is hydrogen, C₁-C₄ alkyl, benzoyl, pivaloyl, an active ester group, or an optionally substituted phenyl protecting group; or when X is the group —OR⁵, R¹ and R⁵ are taken together to form a divalent radical selected from the group consisting of —CH₂— and —(CH₂)₂—;
R², R³, and R⁴ are each independently selected from the group consisting of hydrogen, C₁-C₄ alkyl, phenyl, fluoro, chloro, bromo, iodo, and a group —OR⁶ wherein R⁶ is hydrogen, benzoyl, pivaloyl, or an optionally substituted phenyl protecting group.

36. The pharmaceutical composition of claim 30 wherein the compound is a compound of the formula: 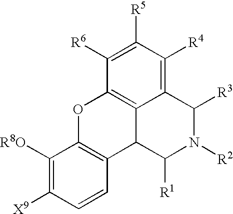
or a pharmaceutically acceptable salt thereof, wherein:
R¹, R², and R³ are each independently selected from the group consisting of hydrogen, C₁-C₄ alkyl or C₂-C₄ alkenyl;
R⁴, R⁵, and R⁶ are each independently selected from the group consisting of hydrogen, C₁-C₄ alkyl, phenyl, halogen, or a group having the formula —OR, where R is as defined above;
R⁸ is hydrogen, C₁-C₄ alkyl, acyl, an active ester group, or an optionally substituted phenyl protecting group;
X⁹ is hydrogen, or a group —OR⁹ wherein R⁹ is hydrogen, C₁-C₄ alkyl, benzoyl, pivaloyl, an active ester group, or an optionally substituted phenyl protecting group; or when X is the group —OR⁹, R⁸ and R⁹ are taken together to form a divalent radical selected from the group consisting of —CH₂— and —(CH₂)₂—.

37. The pharmaceutical composition of claim 30 wherein the compound is a compound of the formula: 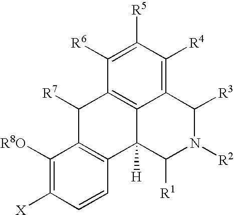
and pharmaceutically acceptable salts thereof, wherein
R¹, R², and R³ are each independently selected from the group consisting of hydrogen, C₁-C₄ alkyl, and C₂-C₄ alkenyl;
R⁴, R⁵, and R⁶ are each independently selected from the group consisting of hydrogen, C₁-C₄ alkyl, phenyl, halogen, and a group having the formula —OR, where R is hydrogen, acyl, an active ester group, or an optionally substituted phenyl protecting group;
R⁷is selected from the group consisting of hydrogen, hydroxy, C₁-C₄ alkyl, C₂-C₄ alkenyl, C₁-C₄ alkoxy, and C₁-C₄ alkylthio;
R⁸ is hydrogen, C₁-C₄ alkyl, acyl, an active ester group, or an optionally substituted phenyl protecting group; and
X is hydrogen, or a group —OR⁹ wherein R⁹ is hydrogen, C₁-C₄ alkyl, benzoyl, pivaloyl, an active ester group, or an optionally substituted phenyl protecting group; or when X is the group —OR⁹, R⁸ and R⁹ are taken together to form a divalent radical selected from the group consisting of —CH₂— and —(CH₂)₂—.

38. The method of claim 19 wherein at least one of R², R³, and R⁴ is other than hydrogen.

39. The method of claim 19 wherein R is hydrogen or methyl; R¹ is hydrogen; and X is hydroxy.

40. The method of claim 19 wherein R is hydrogen, one of R², R³, and R⁴ is methyl, and the others of R², R³, and R⁴ are each hydrogen, R¹ is hydrogen, and X is hydroxy.

41. The compound of claim 29 wherein each of R¹, R², R³, R⁴, R⁵, and R⁶ is hydrogen, and X is a group —OR⁹ wherein R⁹ is hydrogen, benzoyl, pivaloyl, an active ester group, or an optionally substituted phenyl protecting group.

42. The compound of claim 30 wherein each of R¹, R², R³, R⁴, R⁵, R⁶, and R⁷ is hydrogen, and X is a group —OR⁹ wherein R⁹ is hydrogen, benzoyl, pivaloyl, an active ester group, or an optionally substituted phenyl protecting group.
